SEQUENCE LISTING (10 sequences)

SEQ ID NO: 1 - DNA, 20 bp, Artificial Sequence
  P21 Primer Sense
gctggggatgtccgtcagaa

SEQ ID NO: 2 - DNA, 21 bp, Artificial Sequence
  P21 Primer Antisense
gagcgaggcacaagggtacaa

SEQ ID NO: 3 - DNA, 16 bp, Artificial Sequence
  P53 Primer Sense
ggaggttgtgaggcgc

SEQ ID NO: 4 - DNA, 20 bp, Artificial Sequence
  P53 Primer Antisense
cacgcacctcaaagctgttc

SEQ ID NO: 5 - DNA, 24 bp, Artificial Sequence
  CMys Primer Sense
gtttgctgtggcctccagcagaag

SEQ ID NO: 6 - DNA, 24 bp, Artificial Sequence
  CMyc Primer Antisense
cttcccctaccctctcaacgacag

SEQ ID NO: 7 - DNA, 21 bp, Artificial Sequence
  Cyclin D1 Primer Sense
gaacaaacagatcatccgcaa

SEQ ID NO: 8 - DNA, 19 bp, Artificial Sequence
  Cyclin D1 Primer Antisense
tgctcctggcaggcacgga

SEQ ID NO: 9 - DNA, 20 bp, Artificial Sequence
  Beta-actin Primer Sense
agcacaatgaagatcaagat

SEQ ID NO: 10 - DNA, 20 bp, Artificial Sequence
  Beta-actin Primer Antisense
tgtaacgcaactaagtcata

(19) United States
(12) Patent Application Publication
(10) Pub. No.: US 2014/0371244 A1
(43) Pub. Date: Dec. 18, 2014

(54) METHOD FOR TREATING PROSTATE CANCER BY USE OF PHARMACEUTICAL COMPOSITION CONTAINING A3 ADENOSINE RECEPTOR AGONIST

(71) Applicant: Ewha University - Industry Collaboration Foundation, Seoul (KR)

(72) Inventors: Lak Shin JEONG, Seoul (KR); Sang Kook LEE, Seoul (KR); Hwa Jin CHUNG, Seoul (KR); Hyuk Woo LEE, Swaseong-si (KR)

(73) Assignee: Ewha University - Industry Collaboration Foundation, Seoul (KR)

(21) Appl. No.: 14/194,649

(22) Filed: Feb. 28, 2014

Related U.S. Application Data
(63) Continuation of application No. 13/516,634, filed on Jun. 15, 2012, now abandoned.

(30) Foreign Application Priority Data
Dec. 17, 2009 (KR) 10-2009-0126190
Dec. 17, 2009 (KR) 10-2009-0126195
Feb. 2, 2010 (KR) 10-2010-0009630

(51) Int. Cl. C07D 473/34

(52) U.S. Cl. CPC C07D 473/34
USPC 514/263.23

(57) ABSTRACT

The present invention relates to a method by use of a pharmaceutical composition for preventing or treating inflammatory disease, colorectal cancer and prostate cancer, which contains an A₃ adenosine receptor agonist, 2-chloro-N⁶-(3-iodobenzyl)-4′-thioadenosine-5′-N-methyluronamide (thio-Cl-IB-MECA), N⁶-(3-iodobenzyl)-4′-thioadenosine-5′-N-methyluronamide (thio-IB-MECA), or a pharmaceutically acceptable salt thereof. The pharmaceutical composition of the invention is significantly less toxic than conventional A₃ adenosine agonists, and thus is useful for prevention or treatment of inflammatory disease. In addition, it more selectively inhibits the growth of androgen receptor-dependent or independent prostate cancer cells than other A₃ adenosine receptor agonists and thus is useful for prevention or treatment of colorectal cancer or prostate cancer.

### CROSS-REFERENCE TO RELATED APPLICATIONS

This application is a continuation application of U.S. application Ser. No. 13/516,634, which is a national stage application of International Application No. PCT/KR10/09036, filed on Dec. 16, 2010, which claims the foreign benefit of Korean Application No. 10-2009-0126190, filed Dec. 17, 2009; Korean Application No. 10-2009-0126195, filed Dec. 17, 2009; and Korea Application No. 10-2010-0009630, filed Feb. 2, 2010, the contents of which are incorporated by reference as if fully set forth herein.

### TECHNICAL FIELD

The present invention relates to a pharmaceutical composition containing A₃ adenosine receptor agonist. More particularly, the present invention relates to a pharmaceutical composition for preventing or treating prostate cancer, which contains a selective A₃ adenosine receptor agonist, 2-chloro-N⁶-(3-iodobenzyl)-4′-thioadenosine-5′-N-methyluronamide (hereinafter referred to as “thio-Cl-IB-MECA”) and/or N⁶-(3-iodobenzyl)-4′-thioadenosine-5′-N-methyluronamide (hereinafter referred to as “thio-IB-MECA”), which is effective for the prevention or treatment of inflammatory disease, prostate cancer and colorectal cancer.

### BACKGROUND ART

Inflammation is a defensive reaction that occurs in vivo when body tissue is injured by physicochemical factors such as traumas or burns, or biological factors such as bacteria or viruses. It is characterized by symptoms such as congestion, edema, fever, pain and the like. Biochemically, the term “inflammation” refers to a reactive phenomenon that occurs in vivo, such as the local exudation of antibody, a plasma component (containing chemical substances such as histamine or serotonin) or a tissue fluid in the site of inflammation, the infiltration of leukocytes, or fibrous proliferation for recovery, when proinflammatory factors act in vivo. The pattern of inflammatory reaction varies depending on the kind or amount of proinflammatory factor or the immune state in vivo.

Factors which regulate inflammatory reactions can be broadly divided into substances which increase the permeability of blood vessels, and chemical transmitters which promote leukocyte migration (Rubin. Lippincott Williams and Wilkins, 24-46, 2001). In addition, it is known that inflammation can occur in various organs in vivo, and chronic inflammatory diseases can develop into cancers, because they have a close connection with carcinogenesis (Shacter et al., Oncology, 16, 217-26, 229, 2002; Coussens et al., Nature, 420, 860-7, 2002).

Adenosine is produced by the degradation of intracellular ATP, and when intracellular adenosine is accumulated, it is extracellularly released. It is known that, when pathophysiological processes such as inflammatory disease, ischemic heart disease and tissue injury exist, the metabolism of intracellular ATP becomes active, resulting in an increase in the release of adenosine (Linden et al., Annu Rev Pharmacol Toxicol, 41, 775-787, 2001; Stiles, Clin Res, 38, 10-18, 1990). Adenosine receptors are G-protein binding receptors, and a total of four subtypes of adenosine receptors, including A₁, A_{2A}, A_{2B} and A₃, exist. Among them, A_{2A} and A_{2B} increase cyclic adenosine monophosphate (cAMP), whereas A₁ and A₃ reduce cAMP. Thus, intracellular signaling is influenced by the subtype of adenosine receptor that is expressed (Fredholm B B et al., Pharmacol Rev, 53, 527-552, 2001; Jacobson K A et al., Trends pharmacol Sci, 19, 184-191, 1998).

Adenosine receptors are also expressed in macrophages, and substances which influence the interaction between adenosine and selective adenosine receptors can influence the phagocytosis or NO production of such macrophages and also reduce the expression of inflammatory cytokines such as TNF-α, IL-1β and IL-6 (Hasko Get et al., J Immunol, 157, 4634-40, 1996; Sajjadi F Get et al., J Immunol, 156, 3435-42, 1996). Moreover, in an animal model with induced rheumatoid arthritis that is a typical inflammatory disease, an A₃AR agonist was found to have anti-inflammatory activity which is mediated by NF-κB signaling (Fishman Pet et al., Arthritis Res Ther, 8, R33. 2006; Baharav E et al., J Rheumatol, 32:469-76, 2005). Adenosine receptors are much expressed in various cells, and an A₃AR agonist selective for A₃ adenosine receptor (A₃AR) among several adenosine receptors has a high intrinsic activity of activating A₃ adenosine receptor, compared to other subtype receptor-related agonists (Gao Z G et al., Biochem Pharmacol, 65, 1675-84, 2003). Thus, it is considered that the A₃AR agonist is highly likely to be developed into a drug.

However, known A₃AR agonists such as Cl-IB-MECA should be used at high concentrations in the treatment of inflammatory disease, and thus cause side effects such as cytotoxicity. Thus, the use of such A₃AR agonists has been limited.

Cancer is one of intractable diseases to be overcome, and in the whole world, an enormous investment is being made for the development of agents for treating cancer. In Korea, cancer is the first leading cause of disease-related death, and about 100,000 or more people are annually diagnosed as cancer, and about 60,000 or more people annually die due to cancer. Carcinogens include smoking, UV rays, chemical substances, food, and other environmental factors. However, cancer is caused by various factors, and thus the development of agents for treating cancer is difficult and the effects of the therapeutic agents vary depending on the site of cancer. In addition, substances which are currently used as cancer therapeutic agents are significantly toxic and cannot selectively remove cancer cells. Thus, there is an urgent need to develop less toxic and effective anticancer agents for preventing and treating cancer.

Cancer is also called neoplasia and is generally characterized by “uncontrolled cell growth”. Due to the abnormal growth of cancer cells, a cell mass which is called a tumor is formed and invades the surrounding tissue, and in severe cases, metastasizes to other organs of the body. Cancer is an intractable chronic disease which is not fundamentally cured in many cases even when it is treated by surgical, radiation and chemical therapies. Also, it gives patients pain, and ultimately leads to death. Cancer is caused by various factors which are divided into internal factors and external factors.

Although a mechanism by which normal cells are transformed into cancer cells has not been clearly found, it is known that at least 80-90% of cancers are caused by external factors such as environmental factors. The internal factors include genetic factors and immunological factors, and the external factors include chemical substances, radiations, and viruses. Genes related to the development of cancer include oncogenes and tumor suppressor genes, and cancer develops when a balance between these genes is lost due to the internal or external factors.

The properties of cancer cells are similar to those of normal cells in many respects, and thus it is not easy to remove only cancer cells without damaging normal cells. However, cancer cells have several characteristics which are distinguished from those of general cells. Specifically, (1) the proliferation of cancer cells is not controlled, (2) cancer cells relatively lack the characteristics of differentiation, and (3) cancer cells invade and metastasize to the surrounding tissue. Normal cells proliferate by signaling from growth factors, whereas cancer cells have a low dependency on growth factors, do not undergo contact inhibition of growth by the surrounding cells, and actively metastasize by secreting angiogenic factors. In addition, cancer cells do not differentiate, do not undergo apoptosis or programmed cell death, and are genetically instable. It is known that the genetic instability of cancer cells is very important in the progression of cancer and induces tolerance to chemotherapeutic agents (Folksman et al., Science, 235, 442-447, 1987; Liotta et al., Cell, 64, 327-336, 1991).

Cancers are largely classified into blood cancers and solid cancers and occur almost all areas of the body, including lung cancer, stomach cancer, breast cancer, oral cancer, liver cancer, uterine cancer, esophageal cancer, and skin cancer. National cancer statistics indicate that the increase in the cancer death rate after 1996 was ranked in order of lung cancer, colorectal cancer, prostate cancer and pancreatic cancer. Particularly, colorectal cancer is one of the most frequent cancers worldwide, and in Korea, colorectal cancer accounted for 12.0% of all cancers and showed the third leading incidence of cancer and the fourth leading cancer mortality, and it shows a tendency to increase gradually. Among major cancers in men, cancers which showed the highest increase in cancer incidence are prostate cancer and colorectal cancer, the age-standardized incidence rates of which increased by 74.1% and 50.4%, respectively, in 2005 compared to 1999. In advanced foreign countries, three cancers reported to frequently occur are prostate cancer, colorectal cancer and lung cancer in men and breast cancer, colorectal cancer and lung cancer in women. In view of this fact, in Korea in which the style of living is gradually being westernized, the increases in the incidences of colorectal cancer, prostate cancer and breast cancer are expected to be accelerated. In the case of other diseases, therapeutic technology has developed and people have managed the diseases, and thus the rates of death are decreasing. However, in the case of prostate cancer and colorectal cancer, the incidences thereof increase sharply, and thus studies on the development of drugs for treating these cancers are also actively increasing.

Prostate cells require androgen for their growth, stimulation, function and proliferation, and a lack of androgenic stimulation leads to apoptosis. Thus, any therapy for inhibiting the activity of androgen in prostate cells is called androgen deprivation therapy (ADT). Particularly, a major therapy for treating prostate cancer is complete androgen blockade therapy which comprises inhibiting androgen secretion in testicles by surgical or chemical castration together with inhibiting the activity of androgen in prostate cells using an anti-androgen. This complete androgen blockade therapy allows only androgen-independent cells to grow so that the cells change into androgen-independent prostate cancer cells (hormone-refractory prostate cancer cells) in which cancer progresses even after castration.

A primary method for treatment of colorectal cancer is surgical resection, but the recurrence rate of colorectal cancer after surgical resection reaches 40-60% (Reynolds et al., Drugs, 64, 109-118, 2004). For this reason, adjuvant therapy such as radiotherapy or anticancer chemotherapy is required to extend the survival time and to relieve symptoms and to maintain and improve the quality of life. However, there is no absolute principle for the kind of anticancer chemotherapy drug and the route of administration thereof, and the effect of the drug is not satisfactory (Simmonds et al., BMJ, 321, 531-535, 2000). In addition, the rate of response to anticancer chemotherapy and survival rate greatly differ between colorectal cancer patients (McLeod et al., Br J cancer, 79, 191-203, 1999). Studies on drugs, which target the genetic predisposition of tumors, particularly growth signal transduction, and the microenvironment of tumor cells, have been actively conducted in order to develop agents for treating solid cancers, including colorectal cancer (Rowinsky, Drugs, 605, 1-14, 2000), but satisfactory therapeutic agents have not yet been developed.

The present inventors have conducted long-term studies on adenosine receptors and their agonist for the prevention or treatment of various cancers. Adenosine receptors are G-protein binding receptor, and a total of four subtypes of adenosine receptors, including A₁, A_{2A}, A_{2B} and A₃, exist. Among them, A_{2A} and A_{2B} increase cyclic adenosine monophosphate (cAMP), whereas A₁ and A₃ reduce cAMP. Thus, intracellular signaling is influenced by the subtype of adenosine receptor that is expressed (Fredholm B B et al., Pharmacol Rev, 53, 527-552, 2001; Jacobson K A et al., Trends pharmacol Sci, 19, 184-191, 1998). Adenosine receptors are much expressed in various cells, and an A₃AR agonist selective for A₃ adenosine receptor (A₃AR) among several adenosine receptors has a high intrinsic activity of activating A₃ adenosine receptor, compared to other subtype receptor-related agonists (Gao Z G et al., Biochem Pharmacol, 65, 1675-84, 2003). Thus, it is considered that the A₃AR agonist is highly likely to be developed into a drug. In addition, it is known that, because the activation of A₃AR is involved in inflammatory reactions or immune responses, the A₃AR agonist is effective for inhibiting inflammation-related diseases, such as cardiovascular disease, immune disease, rheumatoid arthritis or colitis, and cancer cells (Merighi S et al., Pharmacol Ther. 100, 31-48, 2003; Baraldi P G et al., Med Res Rev, 20, 103-128, 2000; Liang B T et al., Proc Natl Acad Sci USA, 95, 6995-6999, 1998; Fishman P et al., Anti-cancer Drugs, 13, 437-443, 2000).

### DISCLOSURE

### Technical Problem

The present inventors have conducted extensive studies in order to solve the above-described problems occurring in the prior art, and as a result, have found that thio-Cl-IB-MECA and/or thio-IB-MECA, a selective A₃ adenosine receptor agonist, is highly effective for inflammatory disease even at relatively low concentrations compared to conventional adenosine A₃ receptor agonists, and selectively inhibits the growth of not only androgen receptor-dependent (AR+) LNCaP cells, which are hormone-refractory human prostate cancer cells, but also androgen receptor-independent (AR−) PC-3 cells, and selectively inhibits the proliferation of human colorectal cancer HCT 116 cells, and also has no side effects such as toxicity, and thus it can be used as an anticancer agent component capable of substituting for conventional agents for treating inflammatory disease, prostate cancer and colorectal cancer, thereby completing the present invention.

Accordingly, it is an object of the present invention to provide a pharmaceutical composition for preventing and treating inflammatory disease, prostate cancer and colorectal cancer, which is highly effective even at low concentrations and has no side effects such as toxicity.

### Technical Solution

In order to accomplish the above object, the present invention provides a pharmaceutical composition for preventing or treating inflammatory disease, prostate cancer and colorectal cancer, which contains, as an active ingredient, an A₃ adenosine receptor agonist represented by the following formula 1 or a pharmaceutically acceptable salt:

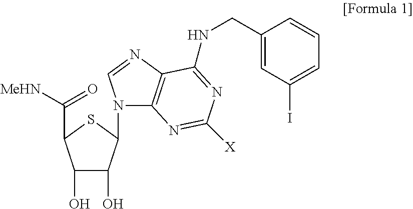

wherein X is Cl or H, and Me is a methyl group.

The above compound thio-Cl-IB-MECA may be prepared by synthesis according to, but not limited to, the following reaction scheme 1 to 3, and may also be synthesized according to a synthesis method modified by a person skilled in the art. The synthesis method according to reaction schemes 1 to 3 is described in detail in U.S. Pat. No. 7,199,127, the entire disclosure of which is incorporated herein by reference.

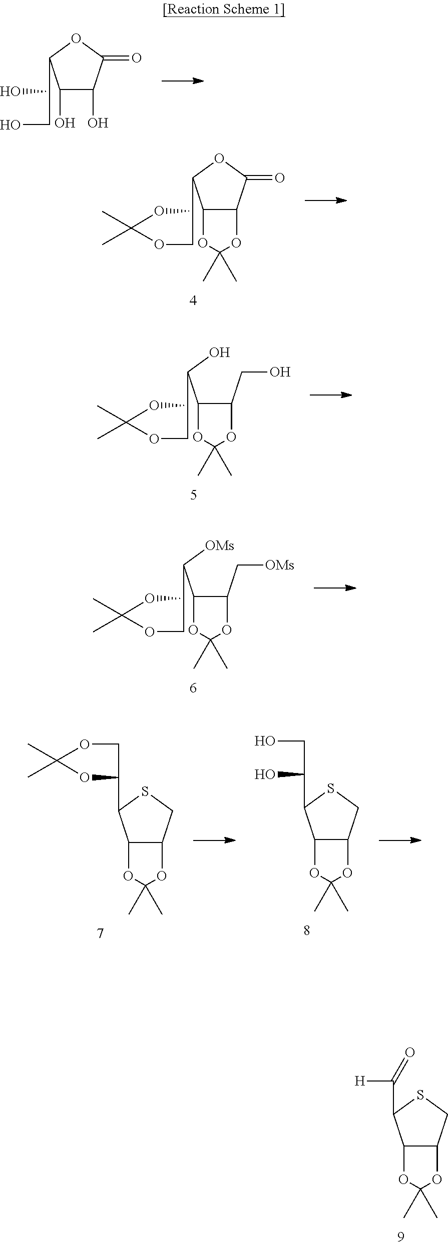

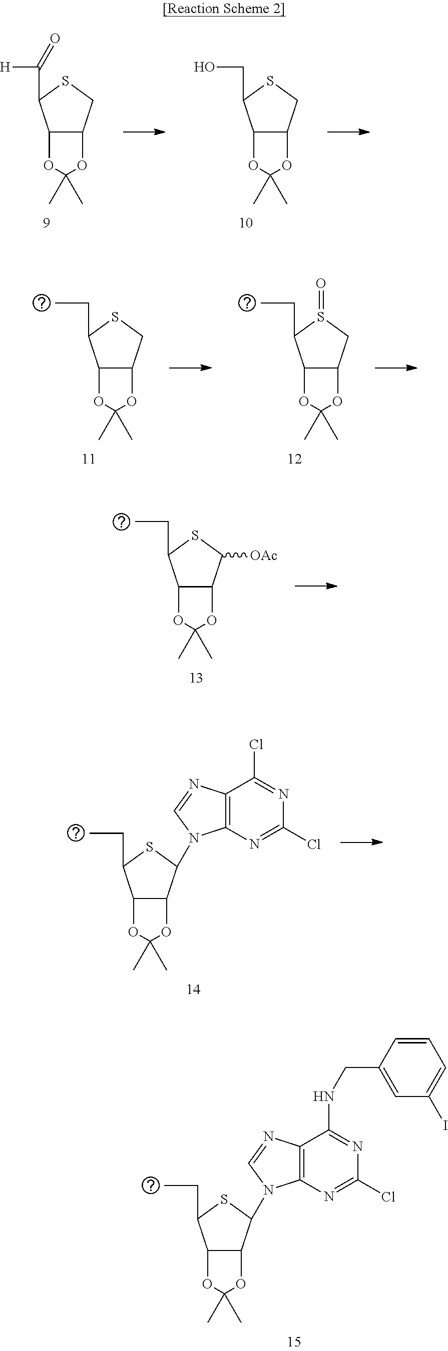

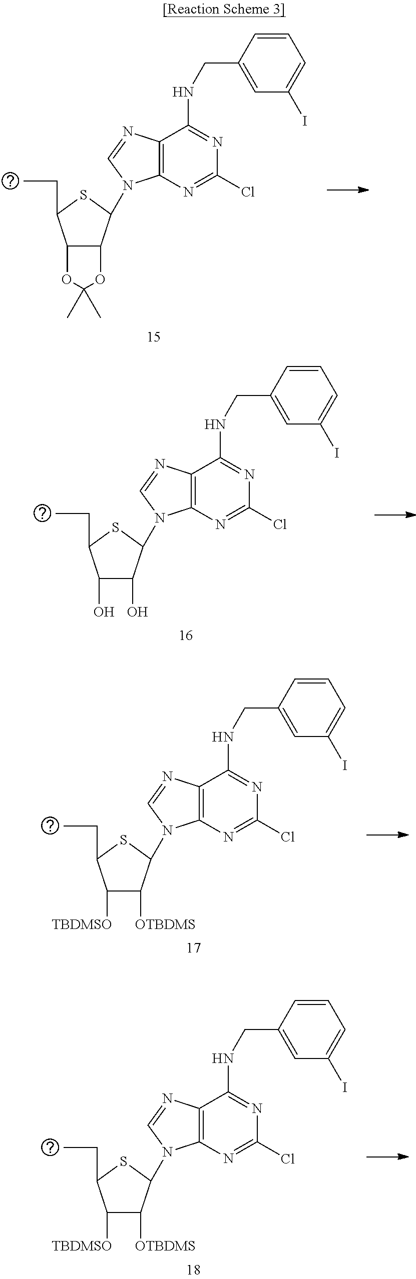 [SMILES: CCC1SC(n2cnc3c(NCc4cccc(I)c4)nc(Cl)nc32)C2OC(C)(C)OC12 ; CCC1SC(n2cnc3c(NCc4cccc(I)c4)nc(Cl)nc32)C(O)C1O ; CCC1SC(n2cnc3c(NCc4cccc(I)c4)nc(Cl)nc32)C(O[Si](C)(C)C(C)(C)C)C1O[Si](C)(C)C(C)(C)C ; CCC1SC(n2cnc3c(NCc4cccc(I)c4)nc(Cl)nc32)C(O[Si](C)(C)C(C)(C)C)C1O[Si](C)(C)C(C)(C)C]

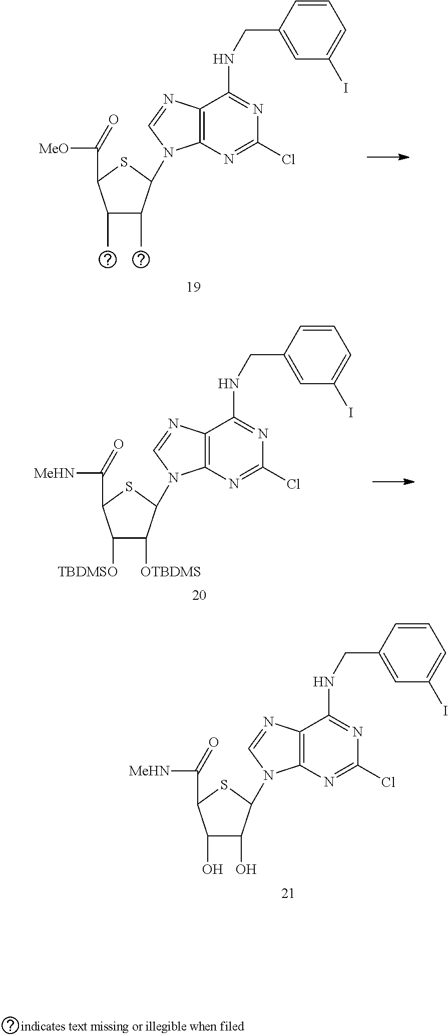 [SMILES: COC(=O)C1SC(n2cnc3c(NCc4cccc(I)c4)nc(Cl)nc32)C(C)C1C ; CNC(=O)C1SC(n2cnc3c(NCc4cccc(I)c4)nc(Cl)nc32)C(O[Si](C)(C)C(C)(C)C)C1O[Si](C)(C)C(C)(C)C ; CNC(=O)C1SC(n2cnc3c(NCc4cccc(I)c4)nc(Cl)nc32)C(O)C1O]

The A₃ adenosine receptor agonist according to the present invention is selected from among thio-Cl-IB-MECA (X in formula 1 is Cl), thio-IB-MECA (X is H), and a mixture thereof.

Examples of pharmaceutically acceptable salts in the present invention include organic addition salts of thio-Cl-IB-MECA or thio-IB-MECA formed with acids, which form a physiological acceptable anion, for example, tosylate, methanesulfonate, malate, acetate, citrate, malonate, tartarate, succinate, benzoate, ascorbate, α-ketoglutarate, and α-glycerophosphate. Suitable inorganic salts may also be used, including hydrochloride, sulfate, nitrate, bicarbonate, and carbonate salts. Pharmaceutically acceptable salts may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example, calcium) salts of carboxylic acids can also be made.

Inflammatory disease which can be treated with the pharmaceutical composition according to the present invention has no concern with the cause of onset and is intended to include diseases mediated by inflammatory processes. Specific examples thereof include sepsis, septic shock, rheumatoid arthritis, osteoarthritis, ankylosing spondylitis, vasculitis, pleurisy, pericarditis, ischemia-associated inflammation, inflammatory aneurysm, nephritis, hepatitis, chronic pulmonary inflammatory disease, bronchitis, nasitis, dermatitis, gastritis, colitis, irritable bowel syndrome, fever caused by infection, and muscular pain.

Sepsis is systemic inflammatory response syndrome (SIRS) caused by bacterial infection. It can be caused by the local or systemic diffusion of toxin by infection and can be induced by microorganisms, such as Staphylococcus, Streptococcus, and Streptococcus pneumoniae. Lipopolysaccharide (LPS) is one of major factors capable of causing sepsis and acts to excite inflammatory reactions by secreting substances that mediate inflammatory reactions. When bacteria invade the body, amplification of LPS-mediated signaling and inflammatory reactions progress, and thus reactions, such as low blood pressure and septic shock, occur due to the activation of vascular endothelial cells and the secretion of nitrogen monoxide (NO) in endothelial cells.

Various factors are involved in inflammatory reactions in inflammatory diseases. The expression of inflammatory reaction-related enzymes and cytokines is regulated mainly by NF-κB. NF-κB is a transcription factor that is present in the form of a homodimer or heterodimer composed of a complex of RelA (p65), RelB, c-Rel, NF-κB 1 (p50) and NF-κB2 (p52), which are the members of the Rel family. Those known as NF-κB are mostly present in the cytoplasm in the form of a heterodimer composed of a RelA/NF-κB 1 (p65/p50) complex.

NF-κB binds to a κB (IκBs) inhibitor in the cytoplasm so that it is present in an inactivated state. When NF-κB is stimulated by LPS or inflammatory substances, it is activated. This process occurs by three substances, IκB kinase (IKK), ubiquitin ligase and 26s proteasome, which regulate IκB. After IκB was phosphorylated by IKK, NF-κB is ubiquitinated by ubiquitin ligase, and finally separated from 26s proteasome and IκB. When free NF-κB moves into the nucleus to bind to a κB-binding site, gene transcription occurs to regulate the expression of enzymes such as iNOS and COX-2, and inflammatory cytokines such as TNF-α and IL-1β, which are involved in inflammation. Thus, the effects of substances for treating inflammatory disease can be analyzed by testing the expression and inhibition of NF-κB, inflammation-related enzymes (iNOS and COX-2) and inflammatory cytokines (TNF-α and IL-1β).

Prostate cancer which can be treated with the pharmaceutical composition of the present invention includes all androgen receptor-dependent or androgen receptor-independent prostate cancers.

Colorectal cancer which can be treated with the pharmaceutical composition of the present invention has no concern with the cause of onset and is intended to include tumors formed in the large intestine, including colon cancer and rectal cancer.

The pharmaceutical composition of the present invention may include at least one selected from among pharmaceutically acceptable carriers or excipients, for example, diluents, lubricants, binders, disintegrants, sweeteners, stabilizers and preservatives, which are conventionally used in the art. It may be formulated in the form of tablets, granules, capsules or powders.

The pharmaceutical composition according to the present invention may be administered intravenously, intraabdominally or orally.

The pharmaceutical composition according to the present invention may contain, in addition to the active ingredient, other known anti-inflammatory drugs or other known anti-tumor drugs.

The dose and number of administrations of the pharmaceutical composition according to the present invention may be suitably determined by a person skilled in the art depending on the patient's age, condition, bodyweight, the severity of disease, the type of drug, the route of administration, and the period of administration. Preferably, the active ingredient of the pharmaceutical composition according to the present invention is administered at a dose of 1-50 mg/kg once or several times a day.

### Advantageous Effects

The pharmaceutical composition according to the present invention exhibits excellent anti-inflammatory effects without side effects such as toxicity even at low concentrations (about ¼) compared to conventional A₃ adenosine receptor agonists.

Moreover, thio-Cl-IB-MECA and thio-IB-MECA show excellent inhibitory effects on the growth of not only androgen receptor-dependent (AR+) LNCaP cells, but also androgen receptor (AR−) PC-3 cells which are human prostate cancer cells, and thus they can be effectively used for the prevention or treatment of prostate cancer. Particularly, the A₃ adenosine receptor agonist according to the present invention has advantages in that it more selectively inhibits the proliferation of prostate cancer cells at low concentrations and shows low toxicity, compared to the conventional A₃ adenosine receptor agonist IB-MECA or Cl-IB-MECA.

In addition, the selective A₃ adenosine receptor agonist thio-Cl-IB-MECA according to the present invention has advantages in that it selectively inhibits the proliferation of colorectal cancer cells and is less toxic.

### DESCRIPTION OF DRAWINGS

FIG. 1 is a set of graphs showing the NO production inhibitory effect (FIG. 1a) and cytotoxic effect (FIG. 1b) of thio-Cl-IB-MECA which is the active ingredient of the inventive composition.

FIGS. 2a and 2b are photographs showing the iNOS protein expression inhibitory effect (FIG. 2a) and iNOS gene expression inhibitory effect (FIG. 2b) of thio-Cl-IB-MECA which is the active ingredient of the inventive composition.

FIGS. 3a and 3b are a graph and a photograph, which show the TNF-α secretion inhibitory effect (FIG. 3a) and TNF-α gene expression inhibitory effect (FIG. 3b) of thio-Cl-IB-MECA which is the active ingredient of the inventive composition.

FIGS. 4a and 4b are photographs showing the IL-1β gene expression inhibitory effect (FIG. 4a) and IL-1β protein expression inhibitory effect (FIG. 4b) of thio-Cl-IB-MECA which is the active ingredient of the inventive composition.

FIG. 5 is a photograph showing the expression inhibitory effect of thio-Cl-IB-MECA, which is the active ingredient of the inventive composition, on the degradation of IκBα protein.

FIG. 6 is a set of photographs showing the results of EMSA carried out to examine the inhibitory effects of thio-Cl-IB-MECA, which is the active ingredient of the inventive composition, on the DNA binding of NF-κB and on the protein binding of p65 which is the subunit of NF-κB.

FIGS. 7a and 7b are photographs showing the results of Western blot carried out to examine the inhibitory effect of thio-Cl-IB-MECA, which is the active ingredient of the inventive composition, on the activation of the NF-κB signaling system in Raw 264.7 cells.

FIGS. 8a and 8b are graphs showing the effects of thio-Cl-IB-MECA, which is the active ingredient of the inventive composition, on survival rate (FIG. 8a) and a change in bodyweight (FIG. 8b) in an animal model with LPS-induced sepsis.

FIG. 9 is a photograph showing the inhibitory effect of thio-Cl-IB-MECA, which is the active ingredient of the inventive composition, on the expression of inflammatory proteins in lung tissue.

FIG. 10 is a set of graphs showing the inhibitory effects of thio-Cl-IB-MECA, which is the active ingredient of the inventive composition, on the growth of (A) LNCaP and (B) PC-3, which are human prostate cancer cells.

FIGS. 11a and 11b are micrographs showing the inhibitory effects of thio-Cl-IB-MECA, which is the active ingredient of the inventive composition, on the growth of LNCaP (FIG. 11a) and PC-3 (FIG. 11b), which are human prostate cancer cells.

FIG. 12 shows the results of cell cycle analysis carried out to examine the inhibitory effects of thio-Cl-IB-MECA, which is the active ingredient of the inventive composition, on the progression of the cell cycle of (A) LNCaP and (B) PC-3, which are human prostate cancer cells.

FIGS. 13a and 13b show the results of Western blot analysis carried out to examine the regulatory effects of thio-Cl-IB-MECA, which is the active ingredient of the inventive composition, on the expression of G1 phase progression inhibition-related factors in LNCaP (FIG. 13a) and PC-3 (FIG. 13b), which are human prostate cancer cells.

FIGS. 14a and 14b show the results of Western blotting analysis carried out to examine the inhibitory effects of thio-Cl-IB-MECA, which is the active ingredient of the inventive composition, on the activation of cell proliferation-related signaling systems in LNCaP (FIG. 14a) and PC-3 (FIG. 14b), which are human prostate cancer cells.

FIG. 15 is a graph showing the effects of IB-MECA, Cl-IB-MECA and thio-Cl-IB-MECA on tumor volume and the inhibition of tumor production in a PC-3 tumor-transplanted animal model.

FIG. 16 is a graph showing the effects of thio-Cl-IB-MECA on tumor volume and the inhibition of tumor production in a PC-3 tumor-transplanted animal model as a function of concentration.

FIG. 17 is a photograph taken 35 days after administration of drugs, which shows tumor volume and the inhibition of tumor production in control animals, and animals administered with IB-MECA, Cl-IB-MECA or thio-Cl-IB-MECA.

FIG. 18 is a graph showing the inhibitory effect of thio-Cl-IB-MECA, which is the active ingredient of the inventive composition, on the growth of human colorectal cancer HCT 116 cells.

FIG. 19 is a micrograph showing the effect of thio-Cl-IB-MECA, which is the active ingredient of the inventive composition, on the growth of human colorectal cancer HCT 116 cells.

FIG. 20 shows the results of cell cycle analysis carried out to examine the inhibitory effect of thio-Cl-IB-MECA, which is the active ingredient of the inventive composition, on the progression of the cell cycle of human colorectal cancer HCT 116 cells.

FIG. 21 shows the results of RT-PCR performed to examine the regulatory effect of thio-Cl-IB-MECA, which is the active ingredient of the inventive composition, on the expression of G1 phase progression inhibition-related factors in human colorectal cancer HCT 116 cells.

FIG. 22 shows the results of Western blot analysis performed to examine the regulatory effect of thio-Cl-IB-MECA, which is the active ingredient of the inventive composition, on the expression of G1 phase progression inhibition-related factors in human colorectal cancer HCT 116 cells.

FIG. 23 shows the results of Western blot analysis performed to examine the inhibitory effect of thio-Cl-IB-MECA, which is the active ingredient of the inventive composition, on the activation of cell proliferation-related signaling systems in human colorectal cancer HCT 116 cells.

FIGS. 24 and 25 are a graph and a tumor photograph, which show the effects of IB-MECA on tumor volume and tumor production inhibitory activity (FIG. 24) and tumors (FIG. 25) in an animal model transplanted with human colorectal cancer HCT 116 cell tumors.

FIGS. 26 and 27 are a graph and a tumor photograph, which show the effects of Cl-IB-MECA on tumor volume and tumor production inhibitory activity (FIG. 26) and tumors (FIG. 27) in an animal model transplanted with human colorectal cancer HCT 116 cell tumors.

FIGS. 28 and 29 are a graph and a tumor photograph, which show the effects of thio-Cl-IB-MECA on tumor volume and tumor production inhibitory activity (FIG. 26) and tumors (FIG. 27) in an animal model transplanted with human colorectal cancer HCT 116 cell tumors.

FIG. 30 shows the changes in bodyweight caused by administration of each of IB-MECA, Cl-IB-MECA and thio-Cl-IB-MECA in an animal model transplanted with human colorectal cancer HCT 116 cell tumors.

### MODE FOR INVENTION

Hereinafter, the present invention will be illustrated with reference to examples. It is to be understood, however, that these examples are not intended to limit the scope of the present invention.

### Test Example 1

The pharmaceutical compositions according to the present invention were tested for anti-inflammatory activities in mouse-derived macrophage RAW 264.7 cells and inflammatory animal models.

### Example 1

### Examination of NO Production Inhibitory Activity (iNOS Assay)

A test was carried out to examine the inhibitory activity of the inhibitory activity of the inventive composition on the enzyme iNOS (inducible nitric oxide synthase) whose expression increases when inflammatory reactions and traumas exist.

Specifically, RAW 264.7 cells (5×10⁵ cells/ml) were added to a 24-well plate with 10% FBS-containing DMEM medium in an amount of 1 ml per well and cultured for 24 hours. After 24 hours, the adherent cells were washed with PBS (phosphate-buffered saline) and then treated with 10% FBS-DMEM medium (phenol red-free) and the inventive composition (containing each of 5, 10 and 20 μM of thio-Cl-IB-MECA). After 30 minutes, 1 μg/ml of LPS was added to the cells which were then cultured in a 5% CO₂ incubator at 37° C. for 20 hours (test groups). As a blank, RAW cells cultured without addition of LPS and the inventive composition were used, and as a control group, RAW cells cultured with addition of only LPS were used.

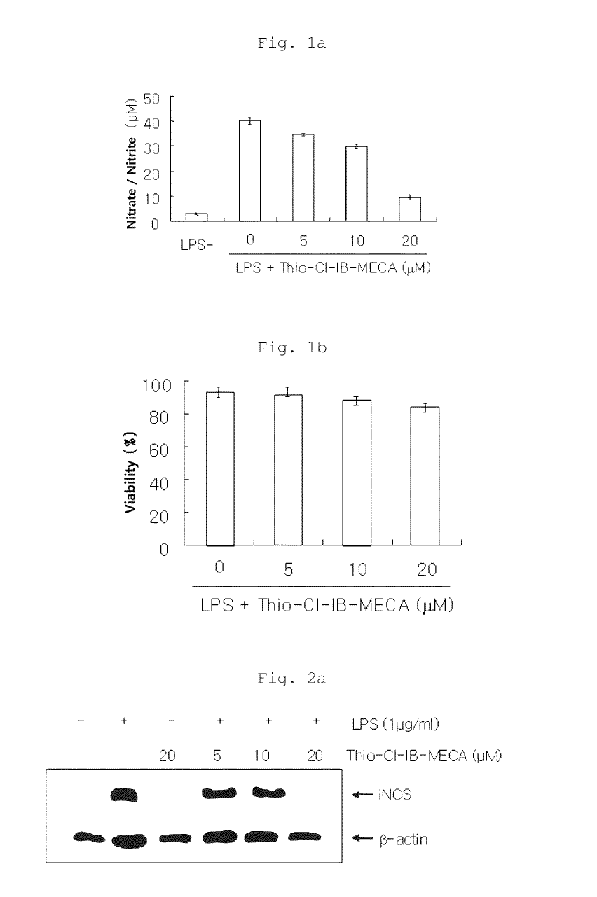

100 μl of the supernatant of each well was taken and allowed to react with 90 μl of each of sulfanilamide solution and N-(1-naphtyl)-ethylenediamine solution. Then, each reaction product was measured for absorbance at 540 nm to determine the amount of nitrate or nitrite produced in the culture medium, and the results of the measurement are shown in FIG. 1.

A standard curve was potted using NaNO₂ solution, and the NO production inhibitory activity of each of the samples was evaluated by calculating NaNO₂ concentration in each test group relative to the control group using the following equation 1 based on the measured absorbance and comparing the calculated NaNO₂ concentration with that in the group treated with LPS alone. Inhibition rate (%) was calculated as nitrate concentration using the following equation, and then the IC₅₀ value of the composition was calculated.

$$Inhibition rate ( \% ) = ( 1 - \frac{( mean value in sample - mean value in L P S - )}{( mean value in L P S + - mean value in L P S - )} ) \times 100 \qquad [ Equation 1 ]$$

As shown in FIG. 1, when the cells were treated with 5, 10 and 20 μM of the inventive composition (thio-Cl-IB-MECA), the production of NO was inhibited in a concentration-dependent manner (see FIG. 1a), and the IC₅₀ value of the composition was 16.23 μM. The IC₅₀ value of the inventive composition was about ¼ of the measured IC₅₀ value (65.7 μM) of the conventional A₃AR agonist IB-MECA, suggesting that the inventive composition has excellent NO production inhibitory activity even at low concentrations.

In order to example whether the iNOS production inhibitory effect is attributable to the toxicity of the sample itself (inventive composition, thio-Cl-IB-MECA), an MTT (3-(4,5-dimethylthiazol-2-yl)-2,5-diphenyltetrazolium bromide) assay was performed. As a result, the RAW 264.7 cells showed a viability of 80% even at 20 μM (the highest concentration of the inventive composition) (see FIG. 1b), suggesting that the effect of the inventive composition is not attributable to toxicity.

### Example 2

### Evaluation of Inhibitory Effects on the Expression of iNOS Protein and Gene

The effects of the inventive composition on the expression of iNOS protein and mRNA were examined by Western blot analysis and RT-PCR. Specifically, RAW 264.7 cells were diluted with 10% FBS-containing medium to a density of 1×10⁶ cells per 100 mm culture dish and cultured under the conditions of 37° C. and 5% CO₂ for 24 hours, followed by washing twice with PBS. The cells were pretreated with each of 20 μM (the highest concentration showing no cytotoxicity), 10 μM and 5 μM of thio-Cl-IB-MECA in 10% FBS-containing medium, and inflammatory reactions in the cells were induced by LPS (1 μg/ml), followed by culture for 18 hours. The non-adherent cells and adherent cells in the cell medium were collected and washed twice with PBS, and then the cells were suspended in lysis buffer and heated at 100° C. for 5 minutes. The cells were cooled and then stored at 20° C. The cells were thawed at 37° C. immediately before use and used in protein quantification and electrophoresis.

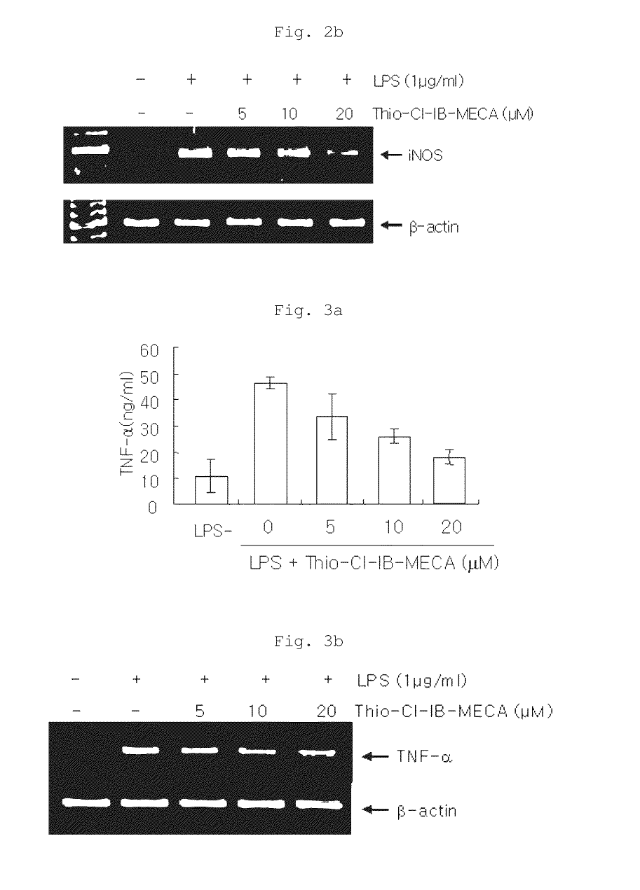

Protein quantification was performed using the BCA method, and 30-50 mg of the protein was electrophoresed on 8-12% SDS-polyacrylamide gel at 150 V for 110 minutes. The gel at the desired site was cut and transferred to a PVDF (polyvinylidene fluoride) for 1 hour, and after which it was washed twice with PBST and stirred in blocking buffer at room temperature for 1 hour. Then, the membrane was washed three times with PBST for 5 minutes each time, after which primary antibody was diluted at a ratio of 1:1,000-1:2,000 with 3% skimmed milk/PBST and was sealed together with the membrane and incubated with stirring at 4° C. for 12 hours or more. The membrane was washed 2-3 times with PBST for 5 minutes each time, after which HRP-conjugated secondary antibody was diluted at a ratio of 1:1,500-1:2,000 and incubated with the membrane at room temperature for 2-3 hours. the membrane was washed three times with PBST for 5 minutes each time and treated with a Western blot substrate (WESTSAVE Up™), and the produced luminescence was examined using LAS-3000. As control groups, a group treated with LPS alone (+control) and a group treated with neither LPS nor the inventive composition (−control) were used to compare protein expression. The results of the comparison are shown in FIG. 2a.

RAW 264.7 cells were diluted with 10% FBS-containing medium to a density of 1×10⁶ cells per 100 mm culture dish and cultured under the conditions of 37° C. and 5% CO₂ for 24 hours, followed by washing twice with PBS. The inventive composition (thio-Cl-IB-MECA) was diluted in 10% FBS-containing medium to concentrations of 20, 10 and 5 μM, and then 10 ml of the culture medium prepared in the 100 mm culture dish was added thereto and cultured for a given time. The non-adherent cells and adherent cells in the cell medium were collected and washed twice with PBS. Then, the cells were lysed with TRI reagent (TRIzol), after which CHCl₃ was added thereto and RNA was extracted from the cells and precipitated using isopropyl alcohol. The RNA precipitate was washed with 70% ethanol and dried in air, after which it was suspended in nuclease-free water. The suspension was heated at 55° C. for 10 minutes and then at 70° C. for 5 minutes, so that the RNA was present as a single chain. The total RNA was quantified with NanoDrop, after which it was diluted to a concentration of 1 μg/μl and made into cDNA using avian myeloblastosis virus (AMV) reverse transcriptase and oligo (dT)₁₅ primers. 0.2 mM dNTP mixture, 10 pmol target gene-specific primer (Table 1) and 0.25 unit Taq DNA polymerase were amplified in GeneAmp PCR system 2400, and then the produced PCR product was electrophoresed on 2% agarose gel at 100 V for 40 minutes and stained with SYBR Safe. The stained DNA was photographed by Alpha Imager, and the photograph is shown in FIG. 2b.

TABLE 1

| Genes | | Sequences |
|---|---|---|
| iNOS | Sense | 5′-ATGTCCGAAGCAAACATCAC-3′ |
| | Antisense | 5′-TAATGTCCAGGAAGTAGGTG-3′ |
| COX-2 | Sense | 5′-CCC CCA CAG TCA AAG ACA CT-3′ |
| | Antisense | 5′-CCC CAA AGA TAG CAT CTG GA-3′ |
| IL-1β | Sense | 5′-TGCAGAGTTCCCCAACTGGTACATC-3′ |
| | Antisense | 5′-GTGCTGCCTAATGTCCCCTTGAATC-3′ |
| TNF-α | Sense | 5′-ATGAGCACAGAAAGCATGATC-3′ |
| | Antisense | 5′-TACAGGCTTGTCACTCGAATT-3′ |
| β-actin | Sense | 5′-TGTGATGGTGGGAATGGGTCAG-3′ |
| | Antisense | 5′-TTTGATGTCACGCACGATTTCC-3′ |

As can be seen in FIG. 2a, the inventive composition (thio-Cl-IB-MECA) inhibited the expression of iNOS protein in a concentration-dependent manner. From the expression of protein in the cells treated with the inventive composition (20 μM of thio-Cl-IB-MECA) and LPS (see the third from the left in FIG. 2a), it could be seen that the expression of iNOS was not attributable to the toxicity of the inventive composition, but was attributable to LPS.

As can be seen in FIG. 2b, the inventive composition (5, 10 or 20 μM thio-Cl-IB-MECA) inhibited the expression of iNOS gene in a concentration-dependent manner compared to the control group treated with LPS (see the second from the left in FIG. 2b).

From such results, it could be seen that the anti-inflammatory activity of the inventive composition (thio-Cl-IB-MECA) directly inhibits the inflammatory reaction-related enzyme iNOS.

### Example 3

### Effects on the Expression of TNF-α

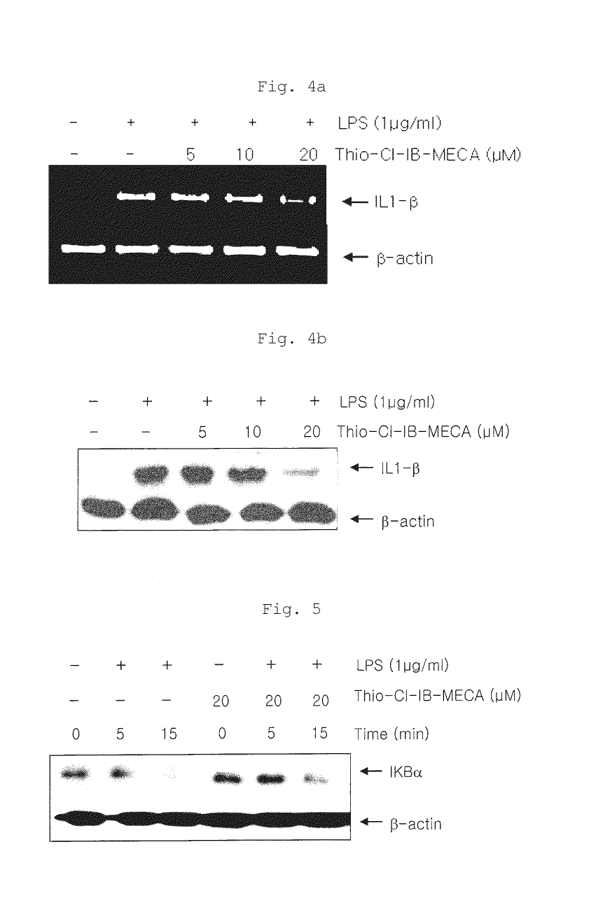

TNF-α is a cytokine whose expression increases in cells stimulated by LPS, and it influences the expression of IL-1β and the progression of inflammatory reactions. Thus, the effect of the inventive composition on the expression of TNF-α was examined. Specifically, RAW 264.7 cells were pretreated with each of 20, 10 and 5 μM of the inventive composition (thio-Cl-IB-MECA) and treated with LPS (1 μg/ml) to induce inflammatory reactions. Then, the cells were incubated for 6 hours, after which the supernatant was taken and the amount of TNF-α secreted into the supernatant was measured using an ELISA (electrophoresis mobility shift assay) kit. The results of the measurement are shown in FIG. 3a.

In addition, in order to examine whether TNF-α is expressed at the gene level, the effect of the inventive composition on the expression of TNF-α mRNA was also tested using the RT-PCR method. The results of the test are shown in FIG. 3b.

As shown in FIG. 3a, the results of ELISA indicated that the amount of TNF-α was concentration-dependently inhibited by treatment with the inventive composition (thio-Cl-IB-MECA). The expression of TNF-α mRNA was also inhibited by the inventive composition (thio-Cl-IB-MECA) (see FIG. 3b).

### Example 4

### Effects on the expression of IL-1β

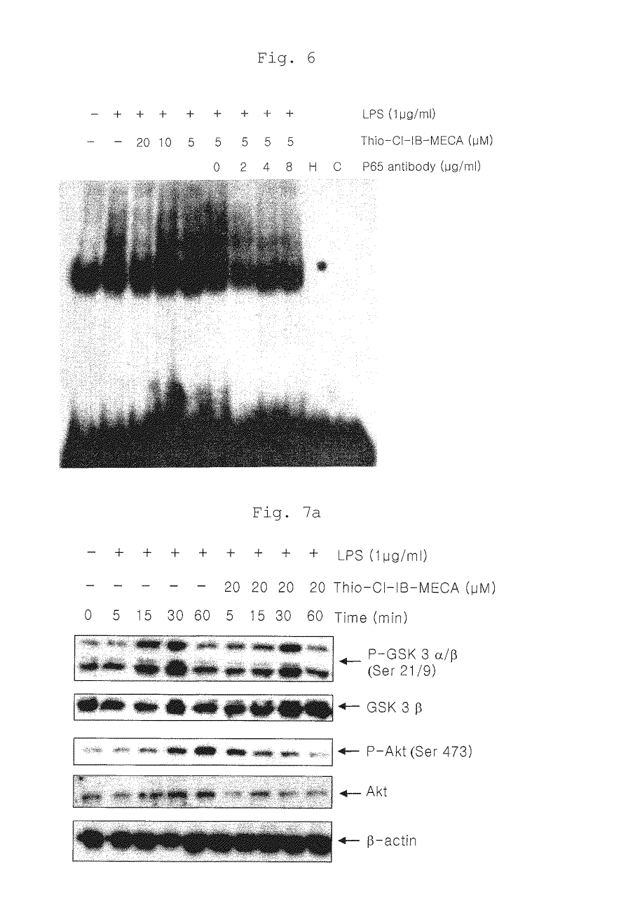

IL-1β is one of major factors involved in inflammation and functions to induce the expression of several genes related to inflammation and tissue injury. Thus, the effects of the inventive composition on the expressions of mRNA gene and protein of IL-1β were tested. Specifically, RAW 264.7 cells were pretreated with each of 20, 10 and 5 μM of the inventive composition (thio-Cl-IB-MECA) and treated with LPS (1 μg/ml) to induce inflammatory reactions, followed by culture for 4 hours. The RNA fraction was collected and cultured for 8 hours, and then the protein was collected and subjected to RT-PCR and Western blot. The results of the analysis are shown in FIGS. 4a and 4b.

The expressions of IL-1β gene (see FIG. 4a) and IL-1β protein (see FIG. 4b) in the test groups treated with the inventive composition (thio-Cl-IB-MECA) were concentration-dependently inhibited compared to those in the control group treated with LPS alone.

### Example 5

### Effect on NF-kB signaling

NF-κB is a transcription factor consisting of p65/p50 and is present in an inactivated state by binding to IκB in the cytoplasm, but when it is stimulated by, for example, LPS, it is activated by a series of processes, including the phosphorylation and degradation of IκB by IκB kinase (IKK). When NF-κB state becomes a free state, it moves into the nucleus and binds to the κB-binding site, thereby regulating the expression of genes, such as iNOS, TNF-α and IL-1β, which are involved in inflammatory reactions. Thus, the effect of the inventive composition on the NF-κB signaling system was tested by Western blot analysis and EMSA.

The test results showed that the degradation of IκB protein in the control group treated with only LPS reached the highest level within 15 minutes (see FIG. 5), whereas IKK in the test group treated with the inventive composition (20 μM thio-Cl-IB-MECA) was inhibited at the same time point, suggesting that the degradation of IκB protein was inhibited (see FIG. 5).

EMSA was performed in order to examine whether NF-κB moves into the nucleus to bind to DNA. As a result, it could be seen that the inventive composition (thio-Cl-IB-MECA) inhibited the DNA binding of NF-κB and the protein bonding of p65 that is the subunit of NF-κB (see FIG. 6).

### Example 6

### Effects on Wnt Protein Expression and β-Catenin Expression

A test was carried out to examine the effects of the inventive composition on the expressions of PI-3 kinase (which influences the NF-κB signaling pathway) and Wnt signaling-related proteins in RAW 264.7 cells. Specifically, RAW 264.7 cells were treated with the inventive composition (20 μM thio-Cl-IB-MECA), and after 30 minutes, were stimulated by LPS. At intervals of 5, 15, 30 and 60 minutes after culture, proteins were extracted from all the cells, and the effects of the inventive composition on the expressions of the proteins were measured in comparison with those in the control group treated with only LPS. The results of the measurement are shown in FIG. 7a. As shown in FIG. 7a, the expression of p-GSK 3α/β phosphorylated by LPS stimulation was inhibited at 30 min and 60 min, and the expression of p-AKT was also inhibited at 30 min and 60 min (FIG. 7a).

In order to examine the intranuclear movement of β-catenin and the accumulation of β-catenin in the cytoplasm, RAW 264.7 cells were treated with each of 5, 10 and 20 μM of the inventive composition (thio-Cl-IB-MECA), and after 30 minutes, the cells were stimulated by LPS. After 1 hour of culture, the cells were collected and separated into a nuclear extract and a cytosol extract. The expressions of β-catenin protein in the cytosol and nuclear portions were analyzed by Western blot, and the results of the analysis are shown in FIG. 7b. As shown in FIG. 7b, the expressions of β-catenin in the cytosol and nuclear portions were concentration-dependently inhibited compared to those in the LPS-treated group. When no LPS stimulation is applied, β-catenin is degraded by phosphorylation. The expression of p-β-catenin in the group treated with the inventive composition (thio-Cl-IB-MECA) was concentration-dependently inhibited compared to that in the group treated with only LPS.

### Example 7

### Animal Model with LPS-Induced Sepsis

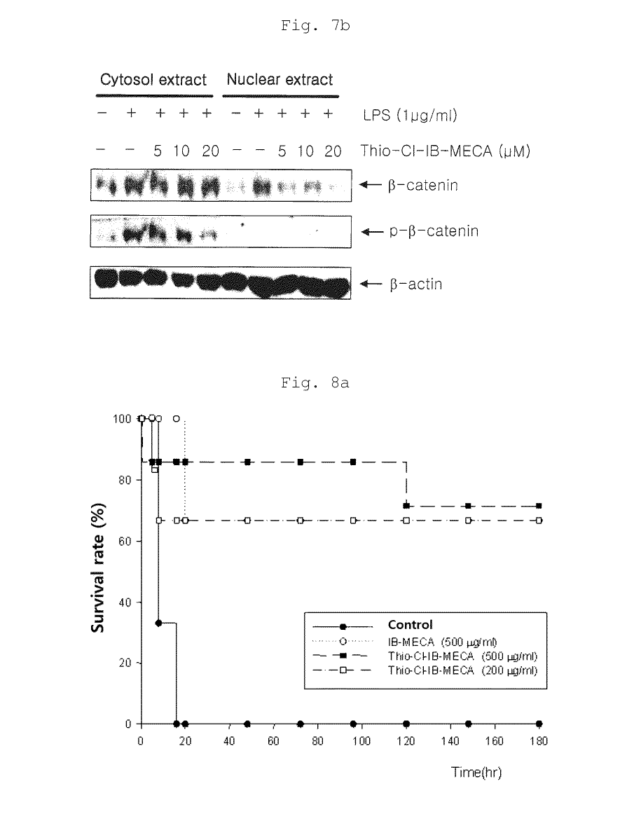

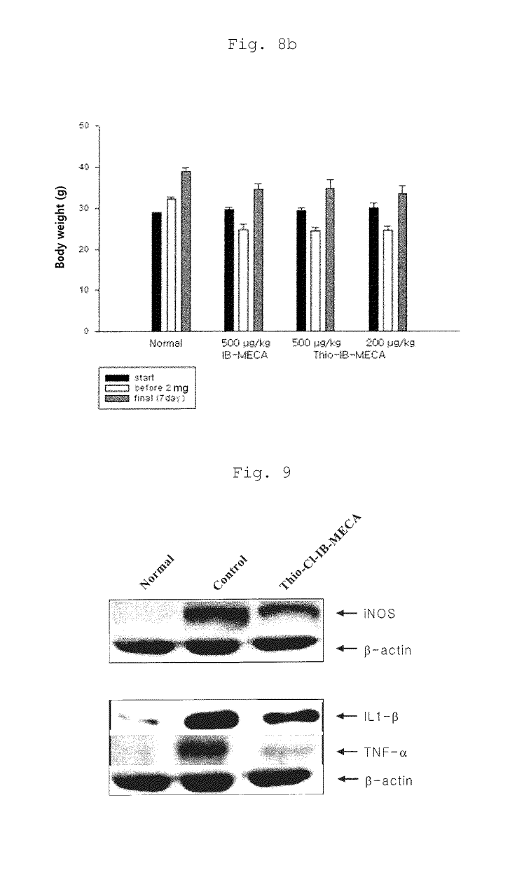

The effect of the inventive composition (thio-Cl-IB-MECA) in an animal model with LPS-induced sepsis was tested. S. marcenes-derived LPS used in the test is known to be strongly pathogenic compared to E. coli-derived LPS. First, LPS was administered at low dose to attenuate an immune response in animals, and after a given time, it was administered at high dose to rapidly induce sepsis and septic shock. Specifically, ICR mice (20-25 g, male) were divided into the following 5 groups, each consisting of 6 mice: a normal group not treated with any substance; a control group administered with only saline; groups administered with the inventive composition (200 and 500 μg/kg of thio-Cl-IB-MECA); and a group administered with the conventional A₃ adenosine receptor agonist (500 μg/kg IB-MECA). 30 minutes administration of these drugs, a solution of 2 mg/kg LPS (S. marcenes) in saline was administered intraabdominally. After 20 hours, the drug was administered again, and 30 minutes, LPS (S. marcenes) was administered again at a dose of 10 mg/kg. The mice were observed hourly up to 6 hours after the second stimulation, and the survival rate was observed up to 7 days after the second stimulation. The effect of the inventive composition (thio-Cl-IB-MECA) was compared with the effect of the prior A₃ adenosine receptor agonist (IB-MECA), and the effects of the inventive composition (thio-Cl-IB-MECA) at different doses (200 and 500 μg/kg) were compared with each other. The results of the test are shown in FIG. 8a.

As shown in FIG. 8a, the survival rates after 1 day were 0% for the control group, 71.4% and 66.7% for the inventive compositions (200 and 500 μg/kg of thio-Cl-IB-MECA), and 66.7% for the prior A₃ adenosine receptor agonist (IB-MECA 500 μg/kg).

The change in bodyweight was measured before the start of the test, before the second administration of the drug, and 7 days after the second administration. As a result, it could be seen that the bodyweight in the normal group continuously increased, but the bodyweight in the groups treated with the drug increased after decreased (FIG. 8b). During the test period, a side effect (a change in bodyweight) caused by administration of the inventive composition (Thio-Cl-IB-MECA) was not observed.

### Example 8

### Expression of Inflammatory Proteins in Lung Tissue

The effects of the inventive composition on the expression of inflammatory proteins in lung tissue were tested. Specifically, alveolar macrophages play an important role in inflammatory reactions caused by infection, and thus the expressions of inflammatory proteins in the macrophages were examined.

ICR mice (25-30 g, male) were divided into the following 3 groups each consisting of 5 mice: a normal group not treated with any substance; a control group administered with only saline; and a group administered with the inventive composition (500 μg/kg thio-Cl-IB-MECA). 2.5 mg/kg of LPS (E. coli) was administered intraabdominally, and after 8 hours, the lung tissue was extracted from the mice. Then, proteins were isolated from the lung tissue, and the expressions of iNOS, TNF-α and IL-1β were analyzed by Western blot. The results of the analysis are shown in FIG. 9.

As can be seen in FIG. 9, the expressions of these proteins in the group treated with the inventive composition were inhibited.

### Example 9

### Animal Toxicity Test

In order to examine the toxicities of thio-Cl-IB-MECA and thio-IB-MECA, each of the compounds was administered orally to ICR white mice (n=10, 25 g) at a dose of 1500 mg/kg weight, and the behavioral change and state of the mice were observed.

All the test animals survived healthfully without particular side effects such as a change in bodyweight up to 7 days after administration of the compounds (thio-Cl-IB-MECA and thio-IB-MECA). Thus, the maximum tolerance doses (MTDs) of the compounds thio-C1-IB-MECA and thio-IB-MECA were more than 1500 mg/kg, suggesting that the compounds are safe drugs.

### Test Example 2

### Example 10

### Measurement of Inhibitory Effect on Growth Human Cancer Cells In Vitro (SRB Assay)

In order to examine the inhibitory effects on the growth of the human prostate cancer cells LNCaP (androgen receptor dependent) and PC-3 (androgen receptor independent), a sulforhodamine B (SRB) assay was performed.

10 μL of a solution of thio-Cl-IB-MECA in 10% dimethylsulfoxide (DMSO) was loaded in each well of a 96-well plate in triplicate at concentrations of 50, 25, 12.5 and 6.25 μM, thus making a test plate. Cells were diluted to a density of 6×10⁴ cells/ml with 10% FBS-containing RPMI 1640 medium supplemented with antibiotics-antimycotics, and 190 μL of the cell suspension was added to each well so as to reach a total volume of 200 μL and was cultured in a 5% CO₂ incubator at 37° C. for 3 days. At the same time, 190 μL of the same cell suspension was loaded into each of 16 wells or more of a fresh 96-well plate containing no sample (thio-Cl-IB-MECA) and was cultured in a 5% CO₂ incubator at 37° C. for 30 minutes, thereby determining reference date. After the culture, 50 μL of 50% trichloroacetic acid (TCA) was added to each well, and the cells were fixed by culture at 4° C. for 1 hour. Then, the well plate was washed five times with tap water and dried. 100 μL of 1% acetic acid solution containing 4% sulforhodamine B (SRB) was added to each well to stain the cells, and the well plate was allowed to stand at room temperature for 1 hour. After the well plate has been washed five times with 1% acetic acid and sufficiently dried, 200 μL of 100 mM Tris-base was added to each well, and the bound staining liquid was dissolved and sufficiently shaken in a shaker. Then, the absorbance at 515 nm was measured using an ELISA microplate reader. Based on the measured absorbance, the cell viability of the test group relative to the control group was calculated using the following equation 2, and based on the viability at each concentration, the IC₅₀ value of the sample was calculated using TableCurve program:

$$Cell viability ( \% ) = \frac{absorbance of sample \text{-} treated group}{absorbance of control group} \times 100 \qquad [ Equation 2 ]$$

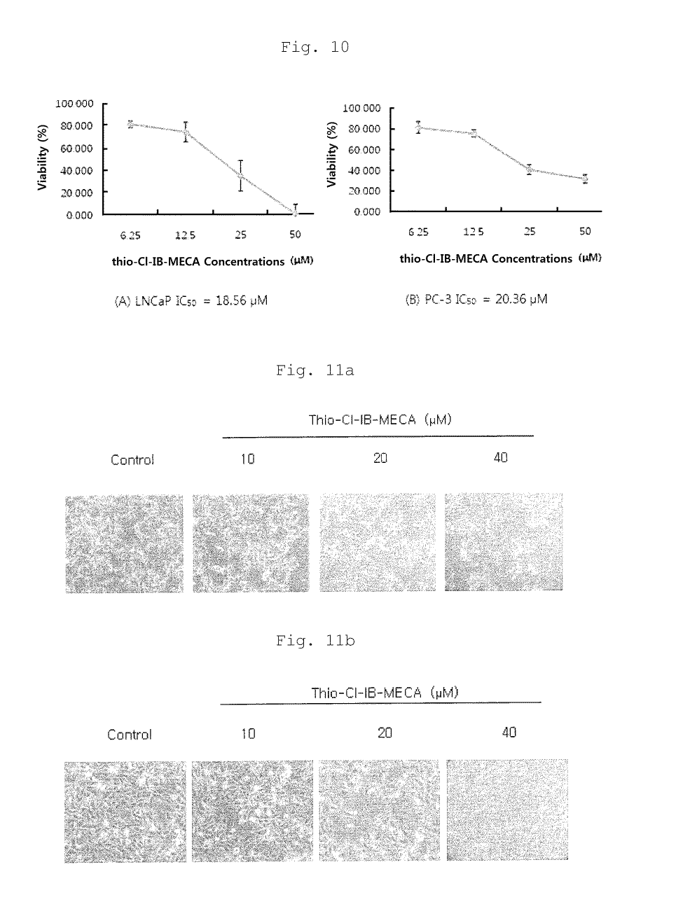

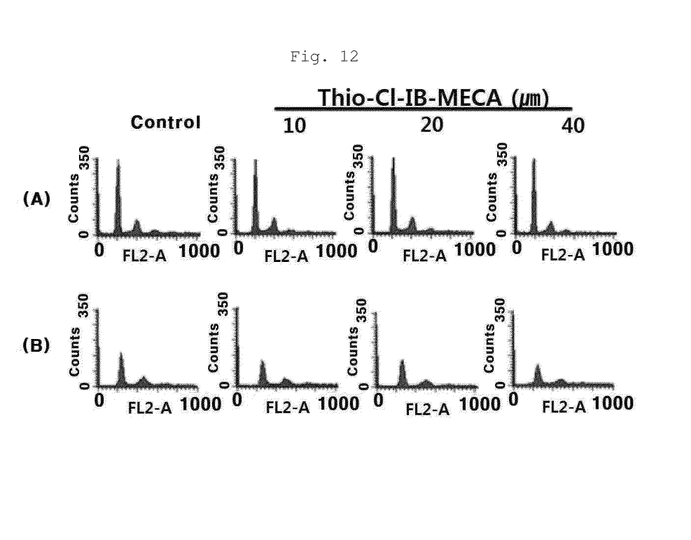

FIG. 10 shows the results of measurement of viability for androgen-dependent human prostate cancer cells LNCaP (A) and androgen-independent human prostate cancer cells PC-3 (B).

For the widely known adenosine derivative IB-MECA, the above test was also carried out to determine the viability of cancer cells, and the IC₅₀ values for each type of prostate cancer cells are shown in Table 2 below.

TABLE 2

| | IB-MECA | thio-Cl-IB-MECA |
|---|---|---|
| LNCap | 54.18 | 18.56 |
| PC-3 | 97.09 | 20.36 |

(IC₅₀ μM)

IB-MECA showed a cell viability of 54% in LNCaP and a cell viability of 63% in PC-3 even at the highest concentration of 50 μM, suggesting that the IC₅₀ value of IB-MECA was 50 μM or higher, but thio-Cl-IB-MECA inhibited the growth of the cells in a concentration-dependent manner even at significantly low concentrations compared to IB-MECA (IC₅₀=18.56 μM; LNCaP cells, IC₅₀=20.36 μM; PC-3 cells) (Table 2 and FIG. 10). Based on the results of the cell inhibitory activity, additional mechanism studies were performed.

### Example 11

### Observation of Morphological Change of Cells In Vitro

Each of LNCaP and PC-3 prostate cancer cell lines was treated with 40, 20 and 10 μM of thio-Cl-IB-MECA and cultured for 48 hours, and then the morphological change of the cells was observed.

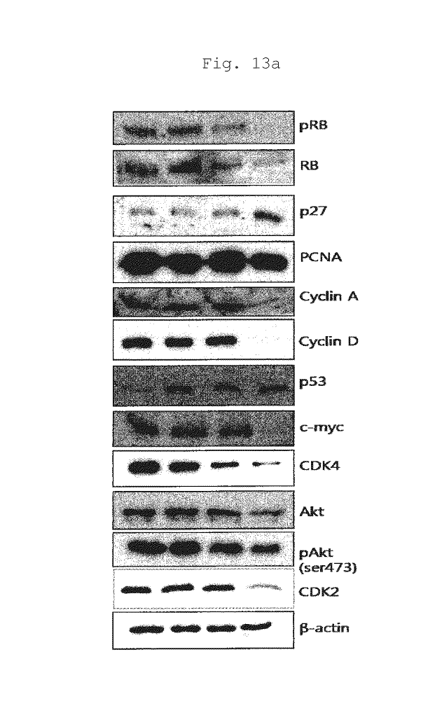

Specifically, each type of the prostate cancer cells was diluted with 10% FBS (Fetal Bovine Serum)-containing medium to a density of 1.0×10⁶ cells per 100 mm culture dish and cultured under the conditions of 37° C. and 5% CO₂, followed by washing twice with PBS (Phosphate Buffered Saline). Thio-Cl-IB-MECA was diluted in 10% FBS-containing medium at required concentration, and then 10 ml of the culture medium prepared in the 100 mm culture dish was added thereto and incubated for a given time. The morphology of the cells was observed as a function of treatment time and concentration (FIGS. 11a and 11b).

As shown in FIGS. 11a and 11b showing the test results, in both the prostate cancer cells LNCaP (FIG. 11a) and PC-3 (FIG. 11b), the control treated with only DMSO without treatment with the sample (thio-Cl-IB-MECA) showed an increase in the number of the cells, but the number of the cells in the group treated with thio-Cl-IB-MECA decreased in a concentration-dependent manner.

### Example 12

### Cell Cycle Analysis In Vitro (FACS)

Cells were treated with 40, 20 and 10 μM of thio-Cl-IB-MECA and cultured for 48 hours, and the cell cycle of the cells was analyzed by flow cytometer analysis (FACS). Specifically, cells were diluted with 10% FBS-containing medium to a density of 1.0×10⁶ cells per 100 mm culture dish and cultured under the conditions of 37° C. and 5% CO₂ for 24 hours, followed by washing twice with PBS. Thio-Cl-IB-MECA was diluted in 10% FBS-containing medium at required concentration, and then 10 ml of the culture medium prepared in the 100 mm culture dish was added thereto and cultured for a given time. The non-adherent cells and adherent cells in the cell medium were collected and washed twice with PBS, after which 500 μL of 100% cold methanol was added thereto and the cells were fixed at 4° C. The cells were washed twice with PBS and allowed to stand in RNase A-containing solution for 30 minutes, after which the cells were stained with propidium iodide (PI) buffer for 5 minutes. After removal of the PI buffer, the cells were transferred into a polystyrene round-bottom tube, and cell cycle analysis was performed by FACScalibur® flow cytometry.

As shown in FIG. 12, in the cases in which LNCaP cells (A) and PC-3 cells (B) were treated with thio-Cl-IB-MECA, an increase in the G₁ phase appeared, and particularly, the G₁ phase arrest in the LNCaP cells appeared clearly compared to that in the PC-3 cells.

### Example 13

### Examination of Regulatory Effects on Expression of Cell Cycle Regulation-Related Proteins and Activation of Signaling System by Western Blot Analysis In Vitro

A test was carried out to examine whether the inhibitory effects of thio-Cl-IB-MECA on the proliferation of LNCaP cells and PC-3 cells are attributable to regulation of the Wnt signaling pathway.

Specifically, each type of LNCaP cells and PC-3 cells was diluted with 10% FBS-containing medium to a density of 1.0×10⁶ cells per 100 mm culture dish and cultured under the conditions of 37° C. and 5% CO₂ for 24 hours, followed by washing twice with PBS. Thio-Cl-IB-MECA was diluted in 10% FBS-containing medium at required concentration, and then 10 ml of the culture medium prepared in the 100 mm culture dish was added thereto and cultured for a given period. The non-adherent cells and adherent cells in the cell medium were collected and washed twice with PBS, after which the cells were suspended in boiling cell lysis buffer and heated at 100° C. for 5 minutes. The cells were cooled and then stored at 20° C., and the stored cells were thawed at 37° C. immediately before use in protein quantification and electrophoresis. Protein quantification was performed using the BCA method, and 30-50 mg of protein was electrophoresed on 8-12% SDS-polyacrylamide gel at 150 V for 110 minutes. The gel at the desired site was cut and transferred to a PVDF (polyvinylidene fluoride) for 1 hour, and after which it was washed twice with PBST and stirred in blocking buffer at room temperature for 1 hour. Then, the membrane was washed three times with PBST for 5 minutes each time, after which primary antibody was diluted at a ratio of 1:1,000-1:2,000 with 3% skimmed milk/PBST, and it was sealed together with the membrane and incubated with stirring at 4° C. for 12 hours or more. The membrane was washed 2-3 times with PBST for 5 minutes each time, after which HRP-conjugated secondary antibody was diluted at a ratio of 1:1,500-1:2,000 and incubated with the membrane at room temperature for 2-3 hours. The membrane was washed three times with PBST for 5 minutes each time and treated with a Western blot substrate (WESTSAVE Up™), and the produced luminescence was examined using LAS-3000.

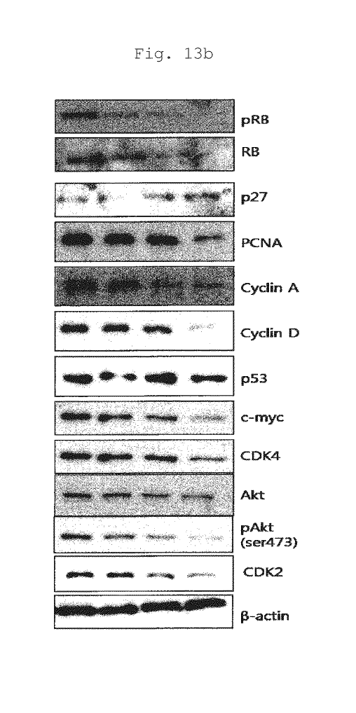

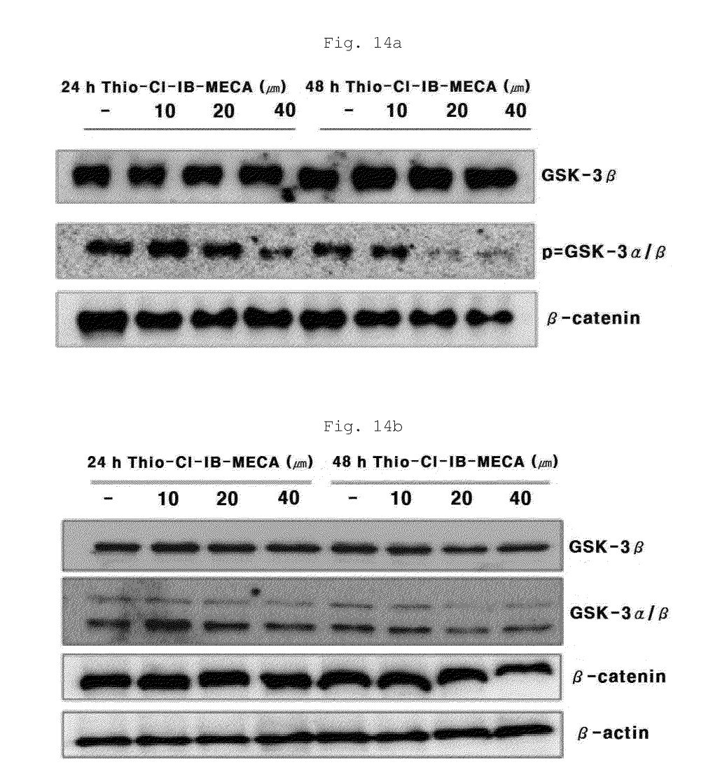

The test results indicated that thio-Cl-IB-MECA increased the expression of the tumor suppressor p53 (which induces G₁ phase arrest) and p27 (which inhibits the cyclin/CDK complex), in the LNCap cells (FIG. 13a) and the PC-3 cells (FIG. 13b), whereas it inhibited the expression and RB phosphorylation of cyclin D, cyclin A, CDK4, c-myc and PCNA. In addition, thio-Cl-IB-MECA inhibited the Wnt signaling pathway (which is a cell proliferation-related signaling system) in the LNCaP cells (FIG. 14a) and PC-3 cells (FIG. 14b).

### Example 14

### Measurement of Anticancer Activity by Animal Test

Based on the anticancer activity of thio-Cl-IB-MECA against prostate cancer cells, proven by the in vitro test, the anticancer activity of thio-Cl-IB-MECA was measured by an animal test. The prostate cancer cell line PC-3 was transplanted subcutaneously into nude mice, and when the tumor size reached 150-200 mm³ after 8 days, the drug thio-Cl-IB-MECA (0.02, 0.2 and 2 mg/kg), thio-IB-MECA (2 mg/kg) or IB-MECA (2 mg/kg) was administered orally every day for 35 days. The tumor size was measured at intervals of 3-5 days. The tumor volume was measured using the following equation 3.

Tumor volume=abc×π/6 [Equation 3]

wherein a represents the longer diameter of the tumor, b represents the shorter diameter of the tumor, and c represents the height of the tumor.

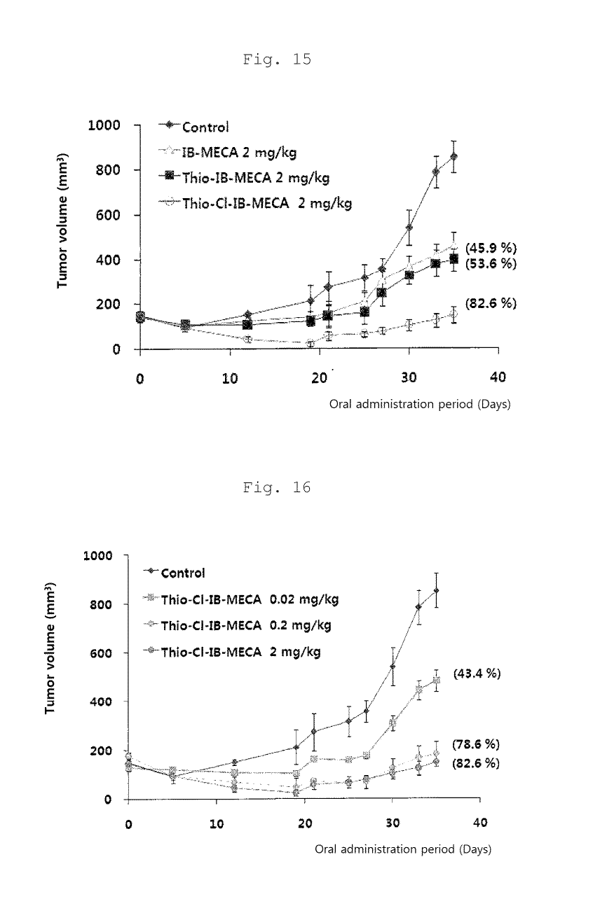

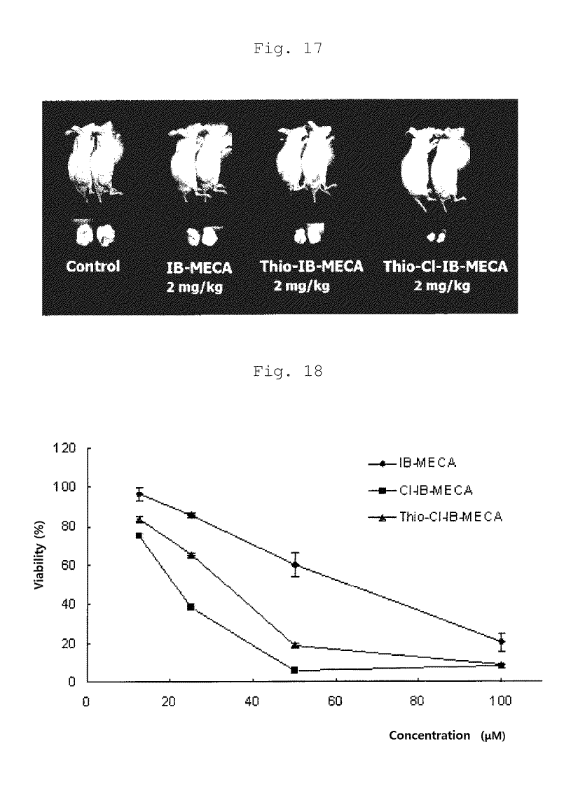

As a result, in the nude mice test animal model transplanted with the cancer cell line PC-3, the three compounds inhibited tumor production in the order of thio-Cl-IB-MECA (∘), thio-IB-MECA (▪) and IB-MECA (▴) at a dose of 2 mg/kg, and the tumor production inhibitory activity values calculated relative to the control group not administered with the active ingredient were 82.6% for thio-Cl-IB-MECA, about 53.6% for thio-IB-MECA and about 45.9% for IB-MECA (FIG. 15). In addition, thio-Cl-IB-MECA inhibited tumor production in a concentration-dependent manner (FIG. 16). FIG. 17 is a tumor photograph of each animal, taken 35 days after administration of the drug.

### Test Example 3

### Example 15

### Measurement of Inhibitory Effect on Growth of Human Cancer Cells In Vitro (SRB Assay)

In order to compare the inhibitory effect of thio-Cl-IB-MECA of the present invention on the inhibition of human colorectal cancer HCT 116 cells with the effects of other A₃ adenosine receptor agonists, IB-MECA and Cl-IB-MECA, a sulforhodamine B (SRB) assay was performed.

Specifically, 10 μL of a solution of thio-Cl-IB-MECA in 10% dimethylsulfoxide (DMSO) was loaded in each well of a 96-well plate in triplicate at concentrations of 100, 50, 25 and 12.5 μM, thus making a test plate. Cells were diluted to a density of 5×10⁴ cells/ml with 10% FBS-containing RPMI 1640 medium supplemented with antibiotics-antimycotics, and 190 μL of the cell suspension was added to each well so as to reach a total volume of 200 μL and was cultured in a 5% CO₂ incubator at 37° C. for 3 days. At the same time, 190 μL of the same cell suspension was loaded into each of 16 wells or more of a fresh 96-well plate containing no sample (thio-Cl-IB-MECA) and was cultured in a 5% CO₂ incubator at 37° C. for 30 minutes, thereby determining reference date. After the culture, 50 μL of 50% trichloroacetic acid (TCA) was added to each well, and the cells were fixed by culture at 4° C. for 1 hour. Then, the well plate was washed five times with tap water and dried. 100 μL of a 1% acetic acid solution containing 4% sulforhodamine B (SRB) was added to each well to stain the cells, and the well plate was allowed to stand at room temperature for 1 hour. After the well plate has been washed five times with 1% acetic acid and sufficiently dried, 200 μL of 100 mM Tris-base was added to each well, and the bound staining liquid was dissolved and sufficiently shaken in a shaker. Then, the absorbance at 515 nm was measured using an ELISA microplate reader. Based on the measured absorbance, the cell viability of the test group relative to the control group was calculated using equation 2 above. Based on the viability at each concentration, the IC₅₀ value of the test sample was calculated using TableCurve program.

In the cases of IB-MECA or Cl-IB-MECA, the viability of cancer cells was examined in the same manner as the case of thio-Cl-IB-MECA. The results of measurement of cancer cell viability are shown in FIG. 18.

As shown in FIG. 18, IB-MECA, Cl-IB-MECA and thio-Cl-IB-MECA concentration-dependently inhibited the growth of the human colorectal cancer HCT 116 cells under the culture condition of 72 hours, and thio-Cl-IB-MECA of the present invention inhibited the growth of the cells at significantly low concentrations compared to the conventional IB-MECA (IC₅₀ of IB-MECA=62.37 μM; IC₅₀ of Cl-IB-MECA=19.26 μM; IC₅₀ of thio-Cl-IB-MECA=37.45 μM) (FIG. 18). Based on the results of the cell inhibitory activity, additional mechanism studies were performed in human colorectal cancer HCT 116 cells.

### Example 16

### Observation of Morphological Cells In Vitro

Human colorectal cancer HCT116 cells were treated with 40 μM of thio-Cl-IB-MECA and cultured for 48 hours, and then the morphological change of the cells was observed.

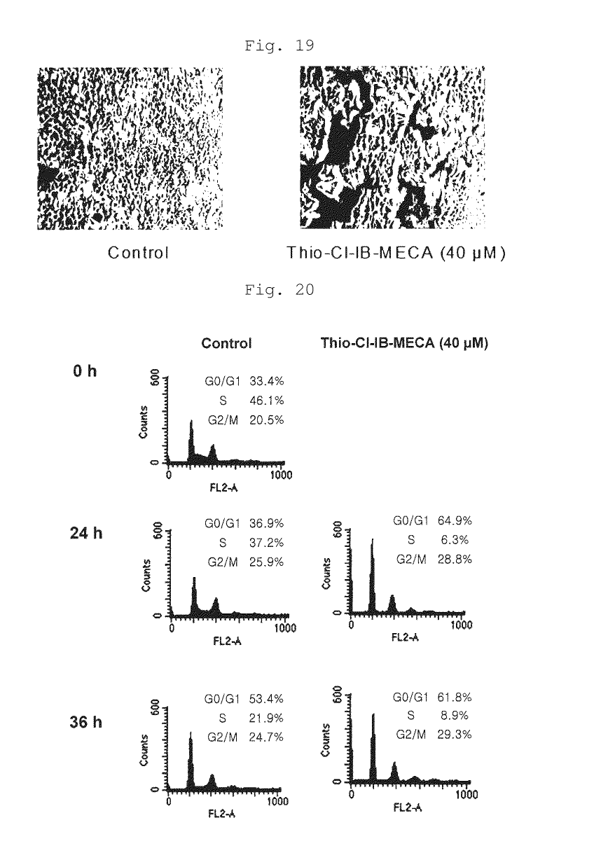

Specifically, HCT116 cells were diluted with 10% FBS (Fetal Bovine Serum)-containing medium to a density of 1.0×10⁶ cells per 100 mm culture dish and cultured under the conditions of 37° C. and 5% CO₂, followed by washing twice with PBS (Phosphate Buffered Saline). Thio-Cl-IB-MECA was diluted to a concentration of 40 μM, and then 10 ml of the culture medium prepared in the 100 mm culture dish was added thereto and incubated for a given time. The morphology of the cells was observed as a function of treatment time and concentration (FIG. 19).

As shown in FIG. 19 showing the test results, the control treated with only DMSO without treatment with the sample (thio-Cl-IB-MECA) showed an increase in the number of the cells, but the number of the cells in the group treated with 40 μM of thio-Cl-IB-MECA decreased.

### Example 17

### Cell Cycle Analysis In Vitro (FACS)

Cells were treated with 40 μM of thio-Cl-IB-MECA and cultured for 24 and 36 hours, and the cell cycle of the cells was analyzed by flow cytometer analysis (FACS). Specifically, cells were diluted with 10% FBS-containing medium to a density of 1.0×10⁶ cells per 100 mm culture dish and cultured under the conditions of 37° C. and 5% CO₂ for 24 hours, followed by washing twice with PBS. Thio-Cl-IB-MECA was diluted in 10% FBS-containing medium to a concentration of 40 μM, and then 10 ml of the culture medium prepared in the 100 mm culture dish was added thereto and cultured for a given time. The non-adherent cells and adherent cells in the cell medium were collected and washed twice with PBS, after which 500 μL of 100% cold methanol was added thereto and the cells were fixed at 4° C. The cells were washed twice with PBS and allowed to stand in RNase A-containing solution for 30 minutes, after which the cells were stained with propidium iodide (PI) buffer for 5 minutes. After removal of the PI buffer, the cells were transferred into a polystyrene round-bottom tube, and the cycle of the cells was analyzed by FACScalibur® flow cytometry.

As shown in FIG. 20, when the human colorectal cancer H116 cells were treated with thio-Cl-IB-MECA, an increase in the G₁ phase compared to that in the control group appeared.

### Example 18

### Examination of Expression of Cell Cycle Regulation-Related Genes by RT-PCR In Vitro

A test was carried out to examine whether cell cycle regulation-related genes involved in the G₁ phase are expressed.

Specifically, HCT 116 cells were diluted with 10% FBS-containing medium to a density of 1×10⁶ cells per 100 mm culture dish and cultured under the conditions of 37° C. and 5% CO₂ for 24 hours, followed by washing twice with PBS. Thio-Cl-IB-MECA was diluted in 10% FBS-containing medium to a concentration of 40 μM, and then 10 ml of the culture medium prepared in the 100 mm culture dish was added thereto and cultured for a given time. The non-adherent cells and adherent cells in the cell medium were collected and washed twice with PBS. Then, the cells were lysed with TRI reagent (TRIzol), after which CHCl₃ was added thereto and RNA was extracted from the cells and precipitated using isopropyl alcohol. The RNA precipitate was washed with 70% ethanol and dried in air, after which it was suspended in nuclease-free water. The suspension was heated at 55° C. for 10 minutes and then at 70° C. for 5 minutes, so that the RNA was present as a single chain. The total RNA was quantified with NanoDrop, after which it was diluted to a concentration of 1 μg/μl and made into cDNA using avian myeloblastosis virus (AMV) reverse transcriptase and oligo (dT)₁₅ primers. 0.2 mM dNTP mixture, 10 pmol target gene-specific primer (Table 3) and 0.25 unit Taq DNA polymerase were amplified in GeneAmp PCR system 2400, and then the produced PCR product was electrophoresed on 2% agarose gel at 100 V for 40 minutes and stained with SYBR Safe. The stained DNA was photographed by Alpha Imager.

TABLE 3

| Genes | | Sequences |
|---|---|---|
| p21 | Sense | 5′-GCT GGG GAT GTC CGT CAG AA-3′ |
| | Antisense | 5′-GAG CGA GGC ACA AGG GTA CAA-3′ |
| p53 | Sense | 5′-GGA GGT TGT GAG GCG C-3′ |
| | Antisense | 5′-CAC GCA CCT CAA AGC TGT TC-3′ |
| CMyc | Sense | 5′-GTT TGC TGT GGC CTC CAG CAG AAG-3′ |
| | Antisense | 5′-CTT CCC CTA CCC TCT CAA CGA CAG-3′ |
| Cyclin D1 | Sense | 5′-GAA CAA ACA GAT CAT CCG CAA-3′ |
| | Antisense | 5′-TGC TCC TGG CAG GCA CGG A-3′ |
| β-actin | Sense | 5′-AGC ACA ATG AAG ATC AAG AT-3′ |
| | Antisense | 5′-TGT AAC GCA ACT AAG TCA TA-3′ |

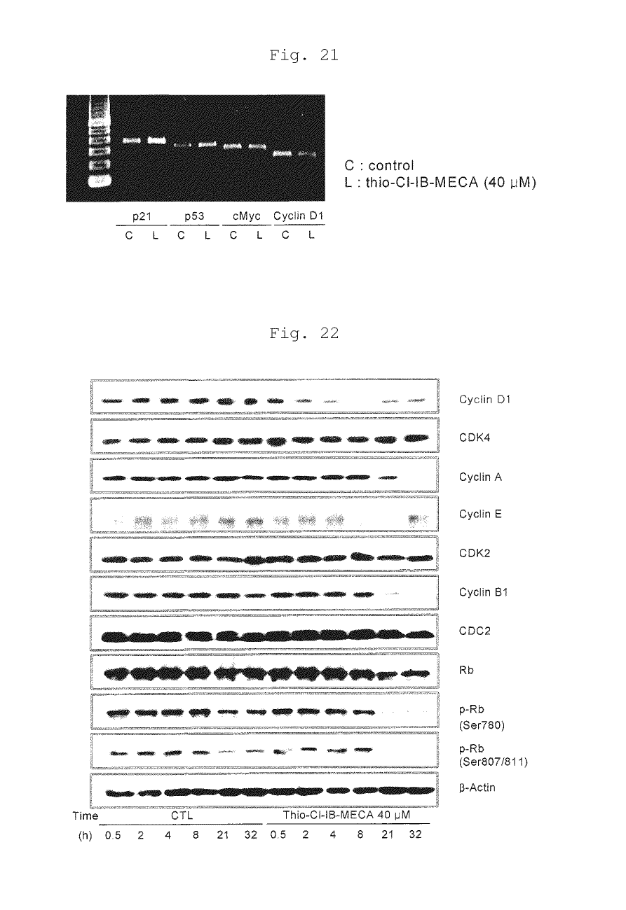

The test results indicated that thio-Cl-IB-MECA increased the expression of the CDK (cyclin-dependent kinase) inhibitor p21 (which induces G₁ phase arrest) and the tumor suppressor p53 in human colorectal cancer HCT 116 cells, whereas it inhibited the expression of cyclin D1 and c-Myc (FIG. 21).

### Example 19

### Examination of Expression of Cell Cycle Regulation-Related Proteins and Activation of Signaling System by Western Blot Analysis In Vitro

Because it was confirmed that thio-Cl-IB-MECA arrests the G₁ phase of human colorectal cancer HCT 116 cells, a test was carried out to examine whether cell cycle regulation-related proteins involved in the G₁ phase are expressed and to examine the expression of Wnt-related proteins in order to examine whether the inhibitory effect of thio-Cl-IB-MECA on the proliferation of cancer cells is attributable to regulation of the signaling pathway.

Specifically, HCT 116 cells were diluted with 10% FBS-containing medium to a density of 1×10⁶ cells per 100 mm culture dish and cultured under the conditions of 37° C. and 5% CO₂ for 24 hours, followed by washing twice with PBS. Thio-Cl-IB-MECA was diluted in 10% FBS-containing medium to a concentration of 40 μM, and then 10 ml of the culture medium prepared in the 100 mm culture dish was added thereto and cultured for a given period. The non-adherent cells and adherent cells in the cell medium were collected and washed twice with PBS, after which the cells were suspended in boiling cell lysis buffer and heated at 100° C. for 5 minutes. The cells were cooled and then stored at 20° C., and the stored cells were thawed at 37° C. immediately before use in protein quantification and electrophoresis. Protein quantification was performed using the BCA method, and 30-50 mg of protein was electrophoresed on 8-12% SDS-polyacrylamide gel at 150 V for 110 minutes. The gel at the desired site was cut and transferred to a PVDF (polyvinylidene fluoride) for 1 hour, and after which it was washed twice with PBST and stirred in blocking buffer at room temperature for 1 hour. Then, the membrane was washed three times with PBST for 5 minutes each time, after which primary antibody was diluted at a ratio of 1:1,000-1:2,000 with 3% skimmed milk/PBST, and it was sealed together with the membrane and incubated with stirring at 4° C. for 12 hours or more. The membrane was washed 2-3 times with PBST for 5 minutes each time, after which HRP-conjugated secondary antibody was diluted at a ratio of 1:1,500-1:2,000 and incubated with the membrane at room temperature for 2-3 hours. The membrane was washed three times with PBST for 5 minutes each time and treated with a Western blot substrate (WESTSAVE Up™), and the produced luminescence was examined using LAS-3000.

As a result, as can be seen in FIG. 22, thio-Cl-IB-MECA inhibited the expression of cyclin D1, cyclin A and cyclin E, which regulate the G₁ phase-to-S phase progression in human colorectal cancer HCT 116 cells, and it also inhibited the expression of the tumor suppressors Rb and p-Rb. Meanwhile, thio-Cl-IB-MECA increased the expression of the tumor suppressor p53 in human colorectal cancer HCT 116 cells and inhibited the Wnt signaling pathway which is a cell proliferation-related signaling system (FIG. 23).

Examples 20 to 22 were carried out in order to compare the anticancer effects of IB-MECA, Cl-IB-MECA and thio-Cl-IB-MECA against human colorectal cancer HCT 116 cells by an animal test.

### Example 20

### Measurement of Anticancer Activity of IB-MECA by Animal Test

HCT 116 cells were prepared at a concentration of 2×10⁶ cells/200 μl (RPMI) and administered subcutaneously into the right frank of 6-week-old female nude mice (Balb/c-nu/nu mice). The tumor size was measured with calipers, and the tumor size reached about 100 mm³, 20 mice having substantially the same tumor size were divided into a control group and three sample-treated groups. The three sample-treated group were administered orally with IB-MECA at doses of 0.02, 0.2 and 2 mg/kg, respectively, for 21 days. In the control group and the sample-treated groups, the bodyweight was measured once every week, and the tumor size was measured at intervals of 3-4 days. The tumor volume was measured using equation 3 above. In the equation, a represents the longer diameter of the tumor, b represents the shorter diameter of the tumor, and c represents the height of the tumor.

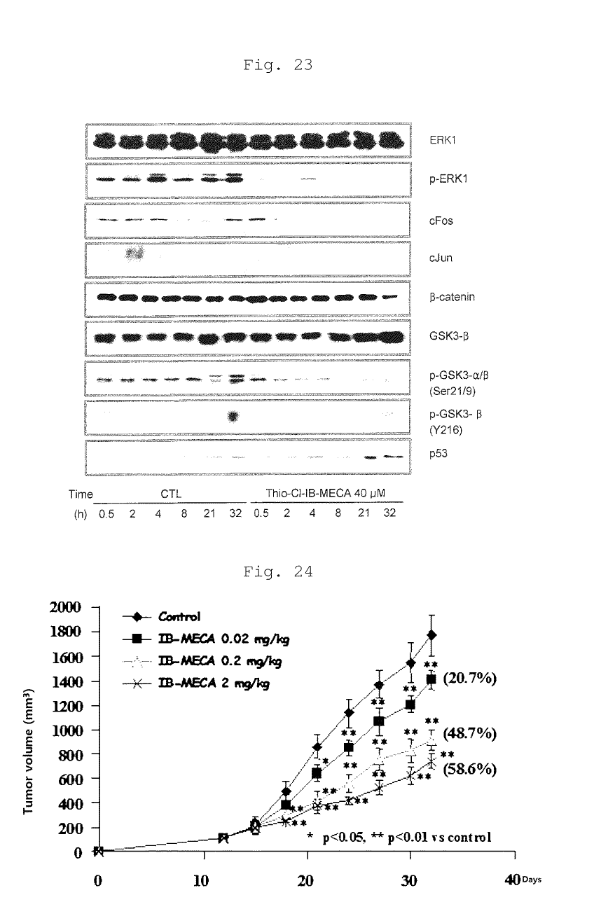

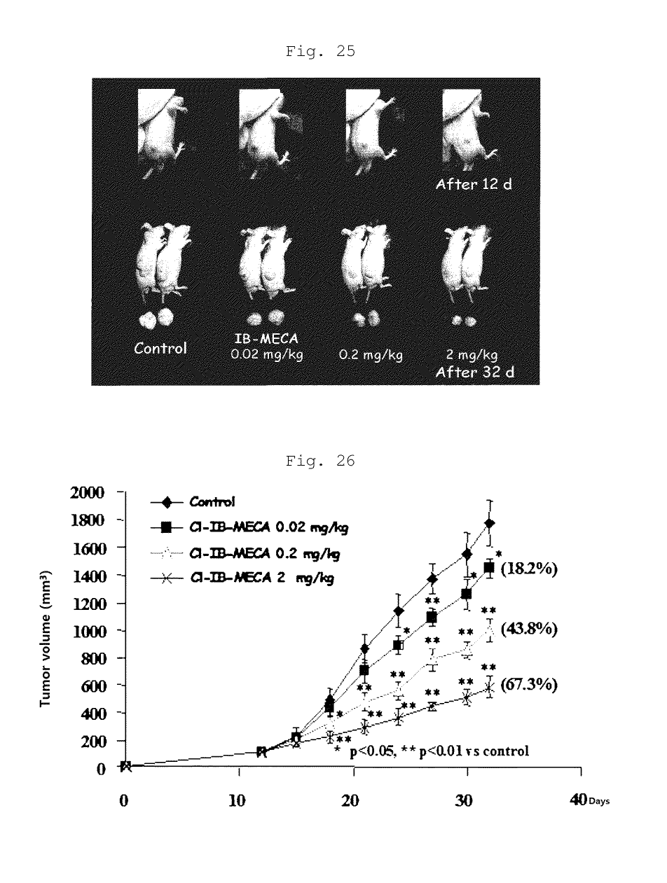

As a result, in the test animal model transplanted with human colorectal cancer HCT 116 cells, IB-MECA showed concentration-dependent inhibition rates of tumor growth of 20.7% at 0.02 mg/kg, 48.7% at 0.2 mg/kg, and 58.6% at 2 mg/kg (FIG. 24). FIG. 25 is a tumor photograph showing the tumor volume and the degree of inhibition of tumor production. During the test period, side effects caused by administration of IB-MECA were not observed.

### Example 21

### Measurement of Anticancer Activity of Cl-IB-MECA by Animal Test

Anticancer activity was measured by an animal test in the same manner as Example 20, except that 6-week-old female nude mice were administered with Cl-IB-MECA in place of IB-MECA as a sample.

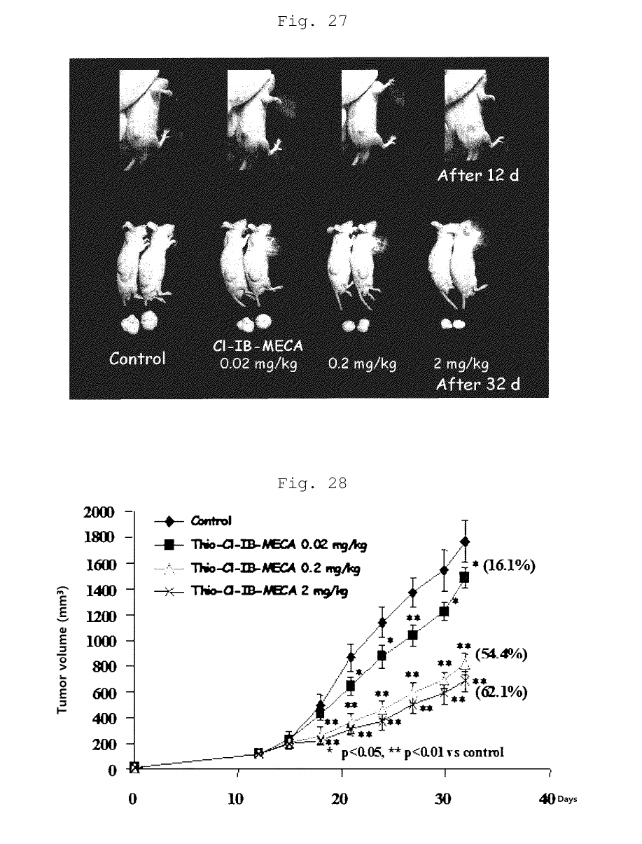

As a result, in the nude mouse test animal model transplanted with human colorectal cancer HCT 116 cells, Cl-IB-MECA showed concentration-dependent inhibition rates of tumor growth of 18.2% at 0.02 mg/kg, 43.8% at 0.2 mg/kg, and 67.3% at 2 mg/kg (FIG. 26). FIG. 27 is a tumor photograph showing the tumor volume and the degree of inhibition of tumor production. During the test period, side effects caused by administration of Cl-IB-MECA were not observed.

### Example 22

### Measurement of Anticancer Activity of Thio-Cl-IB-MECA by Animal Test

Anticancer activity was measured by an animal test in the same manner as Example 20, except that 6-week-old female nude mice were administered with thio-Cl-IB-MECA in place of IB-MECA as a sample.

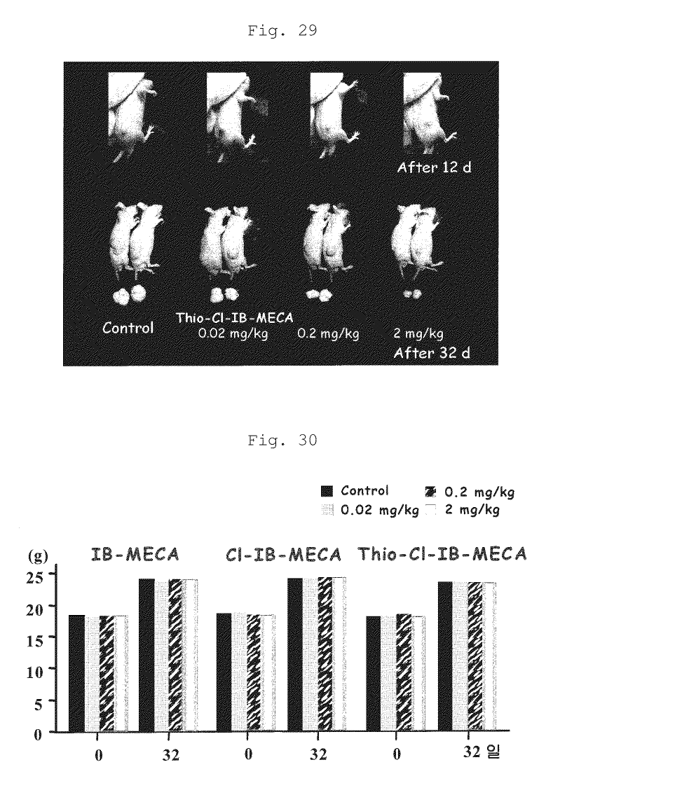

As a result, in the nude mouse test animal model transplanted with human colorectal cancer HCT 116 cells, thio-Cl-IB-MECA showed concentration-dependent inhibition rates of tumor growth of 16.1% at 0.02 mg/kg, 54.4% at 0.2 mg/kg, and 62.1% at 2 mg/kg (FIG. 28). FIG. 29 is a tumor photograph showing the tumor volume and the degree of inhibition of tumor production. During the test period, side effects caused by administration of thio-Cl-IB-MECA were not observed.

Putting the results of Examples 20 to 22, IB-MECA, Cl-IB-MECA and thio-Cl-IB-MECA all showed inhibitory effects on tumor growth in a concentration-dependent manner, and thio-Cl-IB-MECA showed anticancer activity similar to or higher than the conventional IB-MECA or Cl-IB-MECA.

During the test period, the animals survived healthfully without particular side effects (such as a change in bodyweight) caused by administration of IB-MECA, Cl-IB-MECA and thio-Cl-IB-MECA (FIG. 30).

1. A method for treating prostate cancer by use of a pharmaceutical composition comprising, as an active ingredient, an A₃ adenosine receptor agonist represented by the following formula 1 or a pharmaceutically acceptable salt thereof, by administering the active ingredient at a dose of 1˜50 mg/kg once or several times a day: 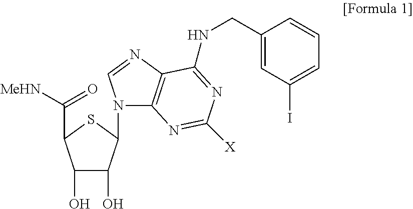
wherein X is Cl or H, and Me is a methyl group.

2. The method of claim 1, the pharmaceutical composition further comprising a pharmaceutically acceptable carrier, excipient, or a mixture thereof.

3. The method of claim 1, wherein the prostate cancer is androgen receptor-dependent prostate cancer or androgen receptor-independent prostate cancer.

4. The method of claim 3, the pharmaceutical composition further comprising a pharmaceutically acceptable carrier, excipient, or a mixture thereof.
